(19) United States
(12) Patent Application Publication
(10) Pub. No.: US 2018/0126067 A1
(43) Pub. Date: May 10, 2018

(54) SYSTEMS AND METHODS FOR COORDINATING AND CONTROLLING INFUSION PUMPS

(71) Applicant: Smiths Medical ASD, Inc., Plymouth, MN (US)

(72) Inventors: Rick LEDFORD, Brooklyn Park, MN (US); Michael WELSCH, Stillwater, MN (US); Larry ZALESKY, Shoreview, MN (US); Eric WILKOWSKE, North Oaks, MN (US); Grant ADAMS, Anoka, MN (US)

(73) Assignee: Smiths Medical ASD, Inc., Plymouth, MN (US)

(21) Appl. No.: 15/569,638

(22) PCT Filed: May 5, 2016
(86) PCT No.: PCT/US2016/030978

§ 371 Date: Oct. 26, 2017

Related U.S. Application Data
(60) Provisional application No. 62/158,213, filed on May 7, 2015.
(60) Provisional application No. 62/332,120, filed on May 5, 2016.

(51) Int. Cl. A61M 5/142; A61M 5/14; A61M 5/172

(52) U.S. Cl. CPC A61M 5/142; A61M 5/16827; A61M 5/172; A61M 5/1407

(57) ABSTRACT

A real-time embedded server system for controlling, in real-time, at least one infusion pump. The embedded server system includes an embedded server including a memory and a processor electrically coupled to the memory and configured to implement a control engine configured to issue control commands to at least one infusion pump, a messaging engine configured to issue messages to and receive messages from at least one infusion pump, an aggregation engine configured to aggregate data related to the operation of the at least one infusion pump, and a networking engine configured to control network access of the embedded server and the at least one infusion pump. A patient care system including at least one embedded server controlling device configured to make patient-specific care decisions and an associated network of devices operably coupled to a patient and configured to be controlled by the at least one controlling device.

### TECHNICAL FIELD

Subject matter hereof relates generally to medical devices, and more particularly, to systems and methods for coordinating and controlling infusion pumps.

### BACKGROUND

Infusion pumps are useful medical devices for providing prescribed fluids, drugs, and other therapies to patients. For example, medications such as antibiotics, chemotherapy drugs, anesthetics, and pain relievers are commonly delivered to patients via infusion pumps, as are nutrients and other supplements. Infusion pumps have been used in hospitals, nursing homes, and in other short-term and long-term medical facilities, as well as for in-home care. Infusion pumps can be particularly useful for the delivery of medical therapies requiring an extended period of time for their administration. There are many types of infusion pumps, including large volume, patient-controlled analgesia (PCA), elastomeric, syringe, enteral, and insulin pumps. Infusion pumps are typically useful in various routes of medication delivery, including intravenously, intra-arterially, intradermally, subcutaneously, intraperitoneally, in close proximity to nerves, and into an intraoperative site, epidural space or subarachnoid space. Traditionally, infusion pumps are locally controlled via the programming of an individual infusion pump, which is generally done on the pump itself. For example, a physician can configure an infusion pump to execute a delivery profile that corresponds to a patient's treatment needs, or a patient can configure an infusion pump according to their individual requirements within pre-defined limits without the involvement of a physician. The physician or patient can program or configure the infusion pump by physical manipulation of the pump interface according to certain physiological, pharmacokinetic, and operational parameters or limits that are often predefined.

Recent developments in computer network technology have led to the incorporation of remote servers that interface with infusion pumps. Traditionally, remote servers are non-real time. That is, a human operator is generally required to be “in the loop” to run the pumps and their associated systems. Although most non-real-time servers can be configured to handle messaging and programming data, they typically do not have quick, reliable response times. Traditional remote servers configured for use with infusion pumps typically do not control the rate of pumping or provide other direct operational control. Rather, traditional remote servers typically provide initial setup or initial configuration programming of the pumps for use. Traditional remote servers can also incorporate pump data for Hospital Information System (HIS) records. Generally, however, traditional remote server control is not reliable enough to control infusion pumps autonomously, due to the uncertainty of the coupled network, inherent latencies in server-network systems, the patient care implications of a remote source of control, as well as other issues, including practice formalities and security concerns. As used throughout this disclosure, the term “remote server” is intended to include external and network-coupled computing devices configured to coordinate and control, in real-time, one or more infusion pumps or associated medical devices for patient care.

Additionally, myriad medical devices are often coupled to a patient for various aspects of patient care. For example, a patient may be coupled to one or more infusion pumps, a pulse oximeter, a blood pressure monitor, and so on. These devices typically function independently of each other and are generally not connected to each other. As a result, patient care is often ad hoc or driven by historical, static protocols. Additionally, International Statistical Classification of Diseases (ICD) codes, the international standard diagnostic tool for epidemiology, health management, billing and insurance coverage determinations, and clinical purposes generally, are often static and assigned after care has been rendered.

Recent advancements in computing technology have led to so-called “smart” devices that include advisory and informational software for clinical decision support such that the devices are no longer “dumb” (and therefore do not require human operator programming as extensively as previous devices). However, putting pump “intelligence” on each device coupled to patients can be prohibitively expensive. Further, it can be un-manageable to have multiple devices communicating with outside networks or outside servers or devices, as this can cause multiple hits on a network, leading to delays and network overloads among other bothersome, inefficient, or even potentially dangerous sources of delay, omission, or error in patient care.

Further, multiple medical data systems pertaining to a single patient (e.g., Electronic Medical Record (EMR), Hospital Information System (HIS), and Information Technology (IT) systems of the various medical devices) are typically not interconnected and function independently of each other to interface with these devices. Such lack of coordination among the systems can also contribute to deleterious increases in practitioner workloads, inefficiencies, and potential dangers to patients as aforementioned. For example, it has been recently observed that a single data system or coordinated multiple data systems that control several infusion pumps for a single patient can be advantageous in cases where a medicament is used to counteract side effects of another medicament. Otherwise, if the deliveries of the medicaments to the patient are being controlled independently, the two medicaments could deleteriously or even dangerously conflict or react with each other.

Therefore, there is a need for systems and methods for coordinating and controlling infusion pumps, such as real-time embedded servers that are configured to coordinate and aggregate one or more infusion pumps. Further, there is a need for systems and methods of an associated network of patient care devices configured to be controlled by one of the networked devices acting as the embedded server.

### SUMMARY

Embodiments described or otherwise contemplated herein substantially meet the aforementioned needs. Embodiments of systems and methods for coordinating and controlling infusion pumps provide real-time embedded servers that are configured to coordinate and aggregate one or more infusion pumps. In an embodiment, a real-time embedded server system includes a server in the same room as the patient (and the one or more pumps), instead of the server being positioned remotely and connected over a network, as in traditional server implementations. Embodiments of the real-time systems overcome the latency and reliability issues of traditional non-real-time servers. Moreover, embodiments provide not just a central control or “brain” for facilitating user interface control of multiple pumps, but rather a system by which an overall patient care suite of devices may communicate and be controlled. Embodiments perform as a real-time system, including status sharing and control. According to embodiments, an embedded device can include any device executable on particular hardware unique to a particular application. Embedded software can comprise control instructions for the particular hardware. In an embodiment, the server is external to the pump or pumps, but is positioned in a rack of pumps. As a result, such a real-time embedded server can coordinate multiple pumps, including, for example, the managing of drug, allergy, and route interactions. Further, handover relay, master-slave, and other pump/infusion interactions can be coordinated. Moreover, the embedded server provides operational control of the pumps coupled by way of a common rack, as well as initial setup and initial configuration programming of the pumps. Embodiments of a real-time embedded server embodied within a rack further provide benefits such as mechanical stability, power distribution, real-time communications, and facilitate communications only between the right pumps, i.e., only between those pumps actually intended for communications. Also in embodiments, it may be advantageous to provide the embedded server externally from the pump or pumps but in a rack of pumps so that for energy considerations (such as, e.g., battery power of a pump or pumps vs. mains power of a rack) the embedded server may be able to have greater processing power and storage capability and therefore be able to perform more complex treatment algorithms. In an embodiment, the embedded server could be specifically implemented in a way such that it would be prevented from becoming or being characterized itself as a medical device, so that security of the embedded server could be more readily and quickly updated as may be desired in a particular care setting.

In an embodiment of an embedded server system for controlling, in real-time, at least one infusion pump, the system comprises a plurality of infusion pumps, wherein each of the infusion pumps includes a pumping mechanism and programmable circuitry configured to receive control commands and control operation of their respective pumping mechanisms. In such an embodiment, each pump could be delivering different infusates to a patient. Alternatively, in such an embodiment some of the pumps could be configured to deliver the same infusate to a patient under the same prescription in an infusion relay scheme or technique. A real-time embedded server includes a memory and a processor electrically coupled to the memory and configured to implement: a control engine configured to issue control commands to at least one infusion pump, the control commands related to the operation of the at least one infusion pump, a messaging engine configured to issue messages to and receive messages from the at least one infusion pump, an aggregation engine configured to aggregate data related to the operation of the at least one infusion pump, and a networking engine; and a network operably coupling the plurality of infusion pumps and the embedded server, wherein the networking engine is configured to control network access of the embedded server and the at least one infusion pump.

In an embodiment, a method for controlling a plurality of infusion pumps with a real-time embedded server comprises implementing a real-time embedded server, the embedded server including a control engine, the control engine configured to issue control commands to at least one infusion pump, the control commands related to the operation of the at least one infusion pump; implementing at least one infusion pump, the at least one infusion pump configured to receive control commands from the embedded server; implementing a network operably coupling the embedded server and the at least one infusion pump; issuing a control command from the control engine to the at least one infusion pump; and executing an operational mode of the at least one infusion pump based on the control command.

In an embodiment, a method of aggregating data related to a plurality of infusion pumps with a real-time embedded server comprises receiving, with a real-time embedded server, a first data from a first infusion pump, the first data comprising data related to the operation of the first infusion pump; storing the received first data; receiving, with an embedded server, a second data from a second infusion pump, the second data comprising data related to the operation of the second infusion pump; storing the received second data; and performing an aggregation algorithm on the stored first data and the stored second data.

In a feature and advantage of embodiments, a system implements a network coupling of the infusion pumps with a real-time server such that the uncertainties of traditional networks are reduced or removed. For example, in a private network of embodiments, the network is not subjected to the interruptions, outside security vulnerabilities, and data overloading of traditionally-networked servers. By implementing a network exclusive for the server and pump, these resources can be focused on their respective pump-specific tasks, possibly without interruption.

In another feature and advantage of embodiments, a system can be implemented with minimal latencies. For example, by utilizing network and server bandwidth for management of infusion pumps, latencies are reduced compared to traditionally-networked servers, which often need to handle non-infusion pump related data and tasks. Further, a dedicated server implemented solely for the management of infusion pumps, as described herein, is less error-prone than traditionally-networked servers.

In another feature and advantage of embodiments, interruptions to routines of medical practitioners are minimized. For example, a practitioner such as an authorized nurse can control all of the pumps for a patient, or a plurality of patients, at a single location from a single point of care. Such a system can be configured to perform similarly to workflows of which the medical practitioner is accustomed, which aids in training and implementation acceptance and compliance.

In another feature and advantage of embodiments, security concerns are reduced compared to traditionally networked servers. In embodiments, a server is implemented on a closed-circuit or private network that is designed to be inaccessible by outside devices and outside networks. Thus, in such embodiments it would be difficult for malicious users to access the server, network, and pumps.

In another feature and advantage of embodiments, a real-time embedded server is configured to coordinate and aggregate multiple types of infusion pumps. For example, in an embodiment, a real-time embedded server can be configured to coordinate Smiths Medical MEDFUSION pumps, which can each have their own controls, protocols, and user interfaces. In embodiments, a real-time embedded server can be configured to coordinate pumps other than Smiths Medical MEDFUSION pumps, which likewise can each have their own controls, protocols, and user interfaces.

In another feature and advantage of embodiments, a real-time embedded server can also be configured for the handling of messaging traffic between the pumps and an external system. For example, the real-time server can coordinate or aggregate communications from the pumps to a Hospital Information System (HIS), or other external system or subsystem. In embodiments, a real-time server can be configured to aggregate data about the infusions from the pumps to the HIS.

In another feature and advantage of embodiments, a real-time embedded server is implemented in a real-time environment. For example, without the aforementioned delays of traditional networks, the server can be immediately responsive, or nearly so, to the infusion pumps to which it is operably coupled and controlling. In embodiments, a real-time embedded server implements a feedback loop from the infusion pumps to which it is operably coupled to provide responsive care to the patient. Algorithms or work flows implemented by the real-time server can evaluate messaging from a respective infusion pump or pumps, inform a medical practitioner of a current status or operational states of the system, or advise the practitioner of recommended action to be taken, or even moderate or change control for those pumps, depending on the status of the system and pumps or condition of the patient.

According to another embodiment, a patient care system comprises at least one controlling device configured to make patient-specific care decisions and an associated network of devices operably coupled to a patient and configured to be controlled by the at least one controlling device.

In another embodiment, an electronic patient care system comprises at least one medical device, an embedded real-time server, and at least one Information Technology (IT) system including at least one of an Electronic Medical Record (EMR) system or a Hospital Information System (HIS), wherein the embedded real-time server provides a communication bridge between the at least one medical device and the at least one IT system so the at least one medical device and the at least one IT system are selectively interconnected to provide inform-and-advise therapy for the patient.

In a feature and advantage of embodiments, adding so-called “intelligence” to a formerly “dumb” device is cost-effective. For example, a network of “dumb” devices can be implemented relatively simply and cheaply according to care needs of patients. One of the devices can be implemented with control logic intelligence for itself and the plurality of dumb devices. Such a system is less costly than a networked system comprised solely of “smart” devices or a system incorporating a costly server to interface with the dumb devices.

In another feature and advantage of embodiments, systems having one control device are simpler to control than traditional systems having multiple devices with control ability. A single control point relieves medical professionals of the burden of recording or “knowing” all of the devices coupled to a patient and the respective progress of each device. Embodiments of the single control device can be configured to automatically evaluate the patient and the devices coupled to the patient, as well as the respective infusions or therapies to the patient.

In a related feature and advantage of embodiments, dynamic control of the plurality of devices coupled to the patient can be implemented. In contrast to traditional systems in which all of the devices function independently of each other and are generally not connected to each other, dynamic control allows for real-time modifications, in an inform-advise context, of recommended changes to therapies or changes to monitoring of the patient among devices coupled to the patient. In an embodiment, all devices can be modified or recommended for modification. In another embodiment, a subset of the coupled devices can be modified or recommended for modification. Embodiments therefore further contrast with traditional systems whereby patient care is ad hoc or driven by historical, static protocols.

In another feature and advantage of embodiments, a single control device is configured to interface with the myriad external medical data systems of a hospital or patient care network. Therefore, data reporting by the control device can facilitate patient care from the external medical data systems. A more streamlined network of systems is therefore attained, as the single control device can be configured to interface with the outside systems through a separate embedded server or directly, instead of each of the patient care devices in the network being configured to interface with each of the outside systems. In addition, by the single control device interfacing to a network or embedded real-time server, instead of multiple devices, the multiple network hits, delays, and network overloads of traditional systems can be avoided.

In another feature and advantage of embodiments, a transition from “dumb” devices to “smart” devices is facilitated by embodiments of control devices and embedded real-time servers as disclosed herein. For example, a server-pump system can be implemented with a real-time server configured for coordinating and controlling a plurality of “dumb” devices in a patient room. Subsequently, should it be desired by the hospital or clinic workflow, one of the “dumb” devices can be retired and replaced with a corresponding control device configured to coordinate and control the rest of the plurality of “dumb” devices. As a result, the discrete real-time server in the patient room can be retired.

The above summary is not intended to describe each illustrated embodiment or every implementation of the subject matter hereof. The figures and the detailed description that follow more particularly exemplify these embodiments.

### BRIEF DESCRIPTION OF THE DRAWINGS

Subject matter hereof may be more completely understood in consideration of the following detailed description of various embodiments in connection with the accompanying figures, in which:

FIG. 1A is a perspective view of an example of a syringe type infusion pump, according to an embodiment.

FIG. 1B is a front view of an example of a control module of an ambulatory type infusion pump, according to an embodiment.

FIG. 2 is a block diagram of an infusion pump system incorporating a real-time embedded server, according to an embodiment.

FIG. 3 is a block diagram of a real-time embedded server system for a plurality of infusion pumps, according to an embodiment.

FIG. 4 is a block diagram of an infusion pump rack system including a real-time embedded server and a plurality of infusion pumps, according to an embodiment.

FIG. 5 is a block diagram of a real-time embedded server, according to an embodiment.

FIG. 6 is a block diagram of a real-time embedded server system and an external hospital information system, according to an embodiment.

FIG. 7A is a block diagram of a real-time embedded server system including messaging traffic between the embedded server and infusion pumps, according to an embodiment.

FIG. 7B is a block diagram of the embedded server of FIG. 7A depicting the storage of the messaging traffic of FIG. 7A, according to an embodiment.

FIG. 8 is a flowchart of a method of controlling a plurality of infusion pumps with a real-time embedded server, according to an embodiment.

FIG. 9 is a flowchart of a method of aggregating data related to a plurality of infusion pumps with a real-time embedded server, according to an embodiment.

FIG. 10 is a block diagram of a system of patient care networks, according to an embodiment.

FIG. 11 is a block diagram of a decentralized embedded server system, according to an embodiment.

FIG. 12 is a flowchart of an inform-advise-control medical device scheme, according to an embodiment.

FIG. 13 is a graph of a medication administration with respect to time in combination with expected and actual patient characteristic values, according to an embodiment.

FIG. 14 is a block diagram of a patient care system including at least one embedded server controlling device, according to an embodiment.

FIG. 15 is a block diagram of example communication interfaces for a patient care system, according to an embodiment.

While embodiments are amenable to various modifications and alternative forms, specifics thereof have been shown by way of example in the drawings and will be described in detail. It should be understood, however, that the intention is not to limit subject matter hereof to the particular embodiments described. On the contrary, the intention is to cover all modifications, equivalents, and alternatives falling within the spirit and scope of subject matter hereof in accordance with the appended claims.

### DETAILED DESCRIPTION OF THE DRAWINGS

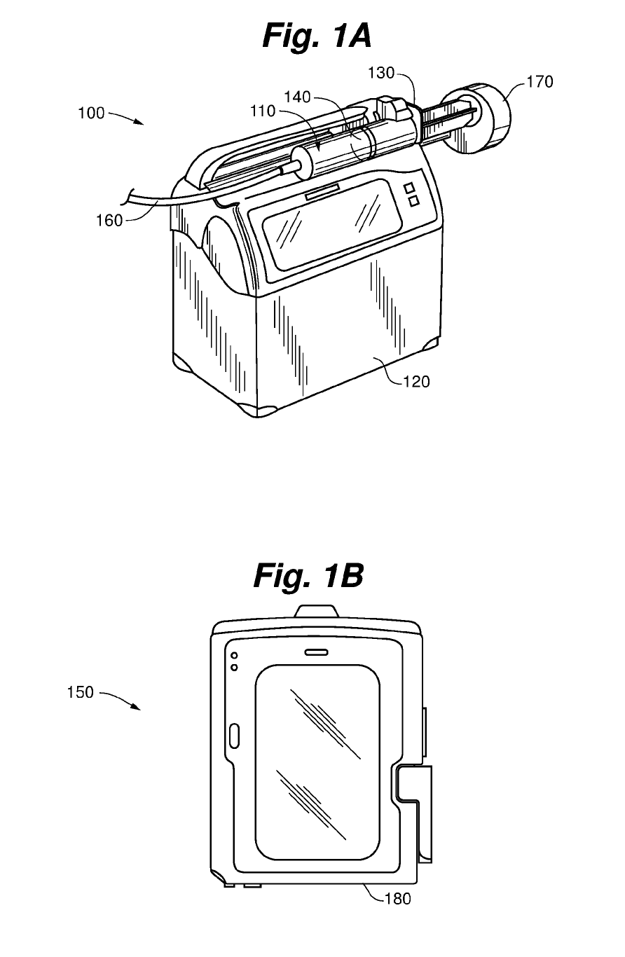

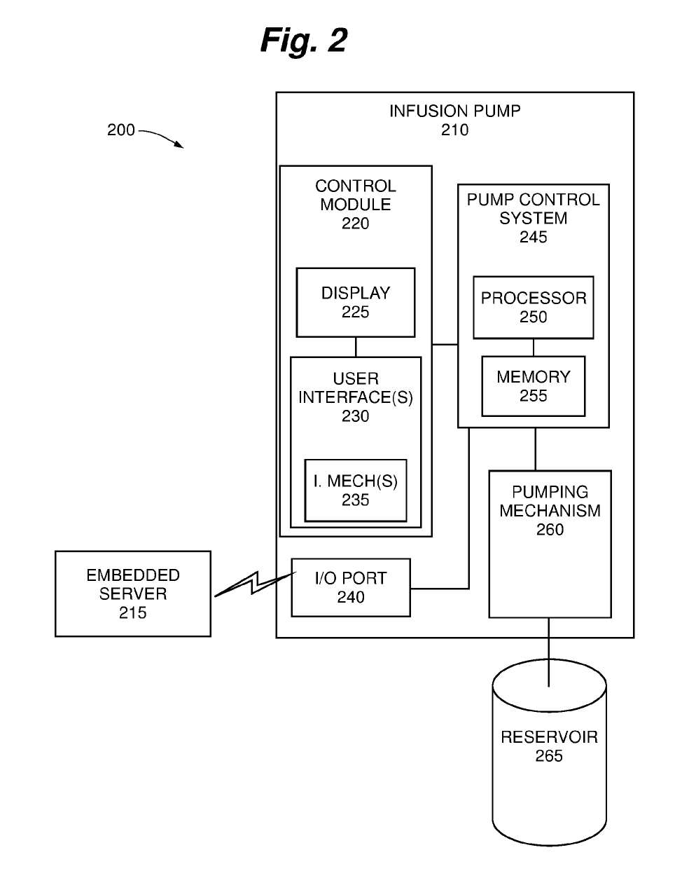

FIGS. 1A and 1B show examples of infusion pumps 100 and 150, respectively (also referred to more generally in this disclosure by numeral 100), which can be used to implement embodiments of the systems and methods discussed herein. In general, infusion pump 100 is a syringe-type pump that can be used to deliver a wide range of infusates, drug therapies and treatments. Infusion pump 100 includes a pharmaceutical container or syringe 110, which is supported on and secured to housing 120 by clamp 130, respectively. In embodiments, syringe 110 can be separately supplied from pump 100. In other embodiments, syringe 110 is an integrated component of pump 100. Syringe 110 includes a plunger 140 that forces fluid outwardly from syringe 110 via infusion line 160 that is connected to a patient. A motor and lead screw arrangement internal to housing 120 of pump 100 cooperatively actuates a pusher or plunger driver mechanism 170, to move plunger 140. In embodiments, a sensor (not shown; which is typically internal to plunger driver mechanism 170) monitors force and/or plunger position in the syringe according to system specifications. In embodiments, plunger position in the syringe could be monitored at, or by way of, a location at or component other than plunger drive mechanism 170.

Infusion pump 150 shown in FIG. 1B is an example of an ambulatory infusion pump control module that can be used to deliver a wide range of infusates, drug therapies and treatments. Such ambulatory pumps can be comfortably worn by or otherwise removably coupled to a user for in-home ambulatory care by way of belts, straps, clips or other simple fastening means, and can also be alternatively provided in ambulatory pole-mounted arrangements within hospitals and other medical care facilities. Infusion pump 150 generally includes a peristaltic type infusion pump mechanism that controls the flow of medication from a reservoir (not shown in FIG. 1B) of fluid coupled to the control module of pump 150 through a conduit from the reservoir which matingly passes along bottom surface 180 of the control module of pump 150. The reservoir can comprise a cassette that is attached to the bottom of pump 150 at surface 180, or an IV bag or other fluid source that is similarly connected to pump 150 via an adapter plate (not shown) at surface 180. Specifically, pump 150 uses valves and an expulsor located on or near bottom surface 180 to selectively squeeze a tube of fluid (not shown) connected to the reservoir to effect the movement of the fluid supplied by the reservoir through the tube and to a patient in peristaltic pumping fashion. Infusion pumps 100 and 150 are two examples of infusion pumps that can be suitable for use with embodiments discussed herein, though other pumps and devices can be used in other embodiments of infusion systems utilizing subject matter hereof.

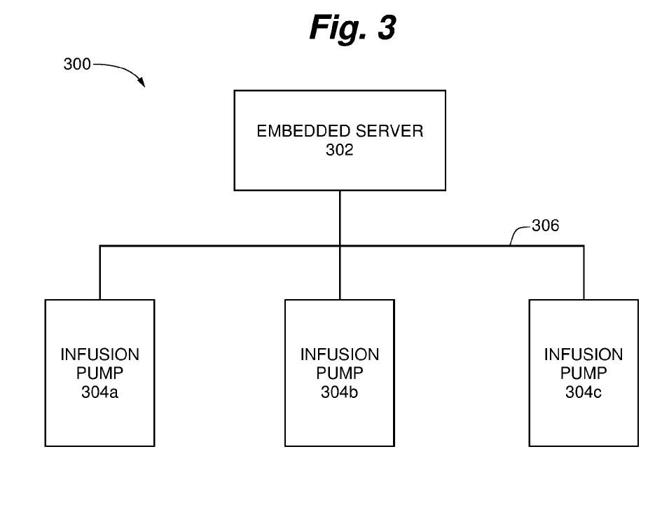

FIG. 2 is a schematic diagram of an example of an infusion pump system 200. System 200 generally includes infusion pump 210 and real-time embedded server 215. In other embodiments, as will be described, multiple infusion pumps 210 can be operably coupled with embedded server 215.

In an embodiment, infusion pump 210 includes pump control system 245 with processor 250 and memory 255 programmable with selected protocols, profiles, segments of profiles, and other settings for controlling operation of pumping mechanism 260 such as, for example, the aforementioned syringe and ambulatory or peristaltic type mechanisms.

In an embodiment, processor 250 can be any programmable device that accepts digital data as input, is configured to process the input according to instructions or algorithms, and provides results as outputs. In an embodiment, processor 250 can be a central processing unit (CPU) configured to carry out the instructions of a computer program. In another embodiment, processor 250 can be an application specific integrated circuit (ASIC). In another embodiment, processor 250 can be a field-programmable gate array (FPGA). Processor 250 is therefore configured to perform basic arithmetical, logical, and input/output operations.

Memory 255 can comprise volatile or non-volatile memory as required by the coupled processor 250 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of subject matter hereof.

Infusion pump 210 can also include control module 220 (e.g., a user interface) for relaying commands to pump control system 245. Control module 220 includes at least one user interface 230 utilizing operator input technology including input mechanism(s) 235, which work with display 225. In some cases display 225 will be considered part of user interface(s) 230. User interface 230 generally allows a user to enter various parameters, including but not limited to names, drug information, limits, delivery shapes, information relating to hospital facilities, as well as various user-specific parameters (e.g., patient age and/or weight). In other embodiments, as will be described, control module 220 can operate in tandem with input, commands, or other programming from embedded server 215. In other embodiments, control module 220 can be bypassed by programming from embedded server 215. In other embodiments, control module 220 can be physically omitted from infusion pump 210 such that control is conducted by embedded server 215 via I/O port 240.

Infusion pump 210 can include a bi-directional serial communications port such as, for example, a USB port, network communications port, or other appropriate interface input/output (I/O) port 240 for connecting infusion pump 210 to embedded server 215 or otherwise establishing a communication path or paths between them. In embodiments, I/O port 240 can be configured for wired or wireless communication, such as by, for example, one or more wired or wireless networking technologies, including Ethernet, one or more of the 802.11 standards, Bluetooth, and ultra-wideband networking technologies. Although not illustrated in FIG. 2, it is to be understood that provision of power to infusion pump 210 can be accomplished by, for example, an AC or DC power cord or source, or an internally provided battery source. Embodiments can also include a wireless power source.

Embedded server 215 comprises a device that is external to infusion pump 210 but configured for communication with infusion pump 210 through I/O port 240. In embodiments, embedded server 215 comprises specialized, non-generic computer software that is configured to interface to a plurality of infusion pumps and/or external systems. For example, embedded server 215 is configured to coordinate infusion pump 210 for pumping operation. In an embodiment, embedded server 215 is further configured to handle messaging traffic between infusion pump 210 and one or more external systems (not illustrated in FIG. 2) such as, for example, a Hospital Information System (HIS) or Electronic Medical Record (EMR) system or between pumps. In embodiments, embedded server 215 is further configured for aggregation of messaging traffic between infusion pump 210 and one or more external systems.

As depicted in FIG. 2, infusion pump 210 is operably coupled to reservoir 265 via pumping mechanism 260. In embodiments, reservoir 265 can comprise any suitable infusate supply, such as an IV bag, syringe, continuous supply, or other infusate storage. In an embodiment, reservoir 265 is coupled to pumping mechanism 260 by cannula or tubing that is suitable for transferring infusate stored in reservoir 265 to pumping mechanism 260. Although not depicted in FIG. 2, it is also to be understood that the contents of reservoir 265 being acted upon by pumping mechanism 260 would then be delivered to a patient by any suitable component such as, for example, an IV infusion line.

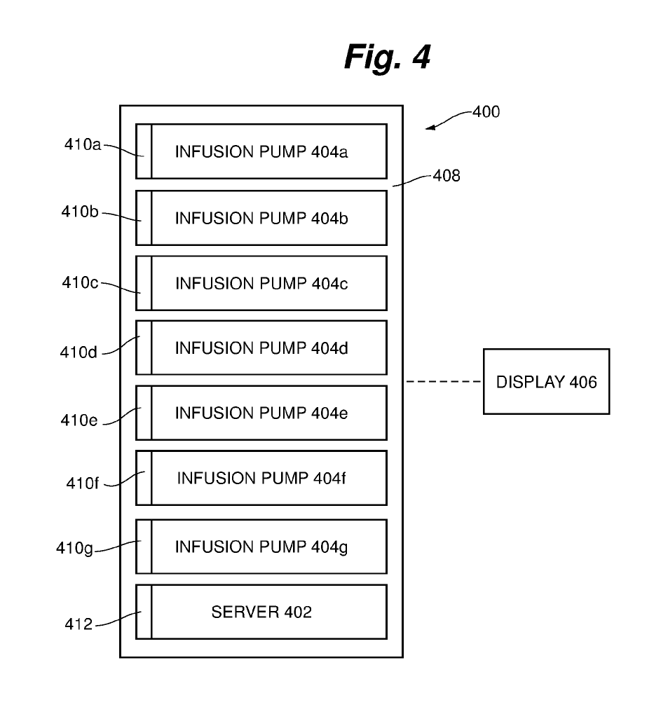

Referring to FIG. 3, a block diagram of an example of a real-time embedded server system 300 for a plurality of infusion pumps 304a-304c is depicted. System 300 generally comprises embedded server 302, a plurality of infusion pumps 304a-304c, and a private network 306. As will be described further below, embedded server 302 generally comprises specialized, non-generic computer software that is configured to interface to a plurality of infusion pumps and/or external systems associated with infusion pumps and/or patient care. In an embodiment, embedded server 302 can be similar in selected features, whether in part or entirely, to embedded server 215. In an embodiment, embedded server 302 or components of embedded server 302 can be embodied within one of infusion pumps 304a-304c. In another embodiment, embedded server 302 can be embodied within another infusion pump (not shown).

Infusion pumps 304a, 304b, and 304c each comprise an infusion pump for providing prescribed fluids, drugs, and other therapies and infusates to one or more patients. For example, in an embodiment, infusion pump 304a can be operably coupled to a first patient. At the same time, infusion pumps 304b and 304c can be operably coupled to a second patient. In another embodiment, infusion pump 304a can be operably coupled to a first patient, infusion pump 304b can be operably coupled to a second patient, and infusion pump 304c can be operably coupled to a third patient, provided that suitable safety concerns are addressed in such an implementation. For example, in some patient care scenarios, it may not be prudent to cross or distribute signals of one particular local subnet of pumps and sensors between separate patients. In another embodiment, all three pumps 304a-304c can be operably coupled to a patient. Thus, embedded server 302 can control any combination of configurations of infusion pumps 304a-304c to one patient or several patients.

In embodiments, one or more infusion pumps 304a-304c can each include their own controls, such as control module 220, referring again to FIG. 2. In such embodiments, the controls of infusion pumps 304a-304c can be bypassed by programming from embedded server 302. In other embodiments, one or more infusion pumps 304a-304c can be implemented without a control module and instead rely on control commands issued by embedded server 302. In other embodiments, one or more infusion pumps 304a-304c can each include their own controls, such as control module 220 in FIG. 2. In such embodiments, the controls of infusion pumps 304a-304c can be bypassed by programming from embedded server 302. In other embodiments, one or more infusion pumps 304a-304c can be implemented without a control module and instead rely on control commands issued by embedded server 302.

Although three infusion pumps 304a, 304b, and 304c are depicted, embodiments of systems as described by example or otherwise contemplated herein can comprise additional or fewer infusion pumps. It is to be appreciated and understood that system 300 can be easily scalable for a plurality of infusion pumps. Further, for example, infusion pump 304a can be of a different type than infusion pump 304b. In other embodiments, all of infusion pumps 304a-304c can be of the same type or different types. Individual pump identification can be conducted by embedded server 302. Further, embedded server 302 can determine a pump type, functionality, and other status, in order to coordinate the care of patient or patients between a plurality of pumps.

In an embodiment, as depicted in FIG. 3, embedded server 302 is operably coupled to infusion pumps 304a-304c via network 306. In an embodiment, for safety and security, network 306 is a closed-circuit, private network that is solely utilized to couple embedded server 302 to infusion pumps 304a-304c. In an embodiment, network 306 can implement any suitable wired or wireless networking technologies, including Ethernet, one or more of the 802.11 standards, Bluetooth, and ultra-wideband networking technologies, among others. In other embodiments, network 306 is separated by hardware or software such that a first portion of network 306 is private to operably couple embedded server 302 to infusion pumps 304a-304c, and a second portion of network 306 is semi-private (but still restricted) to operably couple embedded server 302 to outside networks or systems. In an embodiment, a router or hub can be implemented on network 306 to provide such separation. In an embodiment, network 306 therefore provides a connection between embedded server 302 to interface to and provide real-time control of infusion pumps 304a-304c.

In an embodiment, embedded server 302 can be configured to send one command that is routed by network 306 to all operably coupled infusion pumps. In other embodiments, individual messages or commands are routed to or from specific pumps. For example, embedded server 302 can issue a pump identification request over network 306 to discover the infusion pumps that are connected to that network and thus communicatively coupled. In other embodiments, pump identifications can be transmitted to embedded server 302 over network 306 without a request from embedded server 302. For example, infusion pumps 304a-304c can be configured to transmit identification information once activated on network 306. In embodiments, network 306 can include routing information such that network 306 can forward a particular message or command to a particular infusion pump, once issued by embedded server 302.

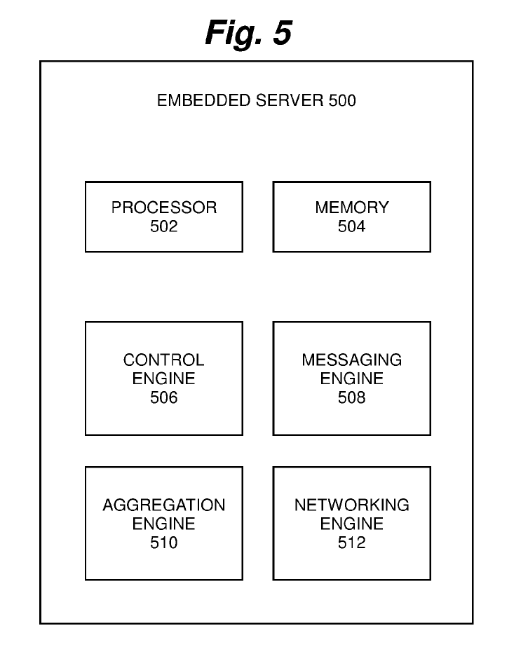

Referring to FIG. 4, a block diagram of an example of an infusion pump rack system 400 including a real-time embedded server 402 and a plurality of infusion pumps is depicted. In an embodiment, infusion pump rack 400 contains, comprises, or is otherwise operatively coupled to server 402 and infusion pumps 404a-404g. It is to be understood that in embodiments, similar to the discussion of embedded server 302 with respect to FIG. 4, in an embodiment, embedded server 402 or components of embedded server 402 can be embodied within one of infusion pumps 404a-404g. In another embodiment, embedded server 402 can be embodied within another infusion pump (not shown).

For example, infusion pump rack 400 generally comprises a body 408, a plurality of pump mounting portions 410a-410g, and at least one server mounting portion 412. As depicted in FIG. 4, pump mounting portion 410a provides an aperture to position and operably couple infusion pump 404a to rack 400. Likewise, pump mounting portion 410b provides an aperture to position and operably couple infusion pump 404b to rack 400. Pump mounting portion 410c provides an aperture to position and operably couple infusion pump 404c to rack 400, and so on with pump mounting portions 410d-410g and infusion pumps 404d-404g, respectively. Such physical and communicative coupling of infusion pumps 404a-404g to or within a single rack 400 allows for infusion pumps 404a-404g to implement specialized and unique infusions, such as piggybacking and takeover modes with respect to each other.

Server mounting portion 412 provides an aperture to position and operably couple server 402 to rack 400. In embodiments, additional server mounting portions can be included for coupling additional servers to infusion pump rack 400. A display 406 can be optionally coupled to infusion pump rack 400. Although not explicitly illustrated in FIG. 4, it is to be understood that in some embodiments, display 406 is electronically coupled to server 402. In embodiments, the electronically coupling of display 406 to server 402 can comprise any suitable communicative software or hardware implementation, such as by hardware wired, wireless including light (infrared), or other suitable coupling. Server 402 can therefore be configured to operationally control all of infusion pumps 404a-404g within infusion pump rack 400; and in some embodiments, such operational control may include user input to, and/or output from, display 406.

Infusion pump rack 400 can further comprise tubing or cannula (not shown) for coupling any of infusion pumps 404a-404g to one or more patients. Such tubing or cannula can further couple any combinations of infusion pumps 404a-404g to each other.

In an embodiment, infusion pump rack 400 can be positioned in a patient's room. In embodiments, infusion pump rack 400 can be positioned at a patient's bedside for bedside control and interaction.

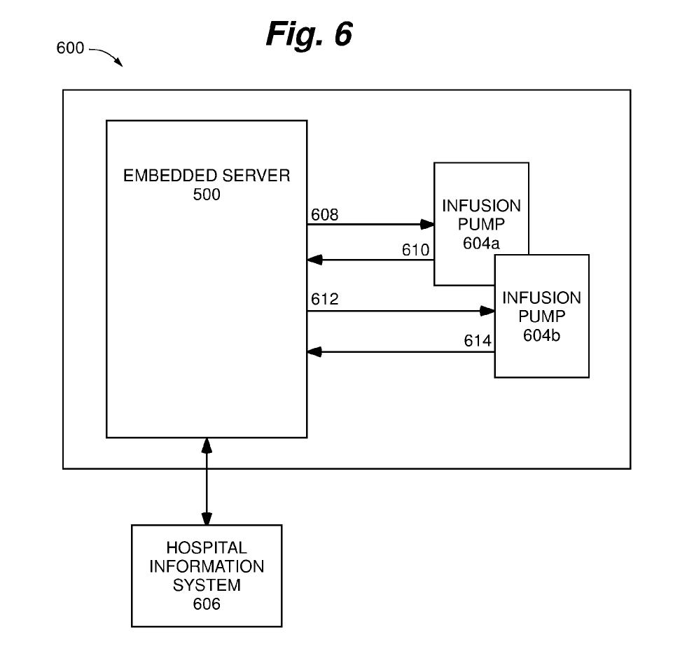

Referring to FIG. 5, a block diagram of an example of a real-time embedded server 500 is depicted, according to an embodiment. Embedded server 500 generally comprises a processor 502 and a memory 504. A plurality of engines can be implemented by or according to processor 502 and memory 504, including a control engine 506, a messaging engine 508, an aggregation engine 510, and a networking engine 512. In embodiments, embedded server 500 can be similar in selected features, whether in part or entirely, to embedded server 215, embedded server 302, and embedded server 402.

In an embodiment, processor 502 can be any programmable device that accepts digital data as input, is configured to process the input according to instructions or algorithms, and provides results as outputs. In an embodiment, processor 502 can be a central processing unit (CPU) configured to carry out the instructions of a computer program. In another embodiment, processor 502 can be an application specific integrated circuit (ASIC). In another embodiment, processor 502 can be a field-programmable gate array (FPGA). Processor 502 is therefore configured to perform basic arithmetical, logical, and input/output operations.

Memory 504 can comprise volatile or non-volatile memory as required by the coupled processor 502 to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. In an embodiment, memory 504 can comprise a database. In an embodiment, memory 504 comprises memory for operation of the processor and a separate database for storing records related to the system. The foregoing lists in no way limit the type of memory that can be used, as these embodiments are given only by way of example and are not intended to limit the scope of the subject matter hereof.

The engines described herein can be constructed, programmed, configured, or otherwise adapted, to autonomously carry out a function or set of functions. The term engine as used throughout this document is defined as a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that cause the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboards, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically embodied configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly in parallel or series with, and/or complementary to other functions, or distributed differently among a set of engines than specifically illustrated in the examples herein.

Control engine 506 is configured to execute the function or set of functions of controlling one or more infusion pumps for operation. In an embodiment, control engine 506 can issue commands to one or more infusion pumps to control the infusion parameters for the one or more infusion pumps. In an embodiment, control engine 506 can receive data, responses, or other information related to the operation of the one or more infusion pumps from the one or more infusion pumps. In embodiments, control engine 506 can utilize the received data, responses, or other information in order to make decisions about infusions. For example, infusion parameters for one or more infusion pumps can be adjusted by control engine 506 after receiving data, responses, or other information from the one or more infusion pumps. A feedback or quasi-feedback system is thereby created by embedded server 500 and the infusion pumps. The feedback thereby occurs when outputs from the one or more infusion pumps are fed back as inputs as part of a chain of cause-and-effect that forms a feedback or quasi-feedback circuit with embedded server 500.

In an embodiment, control engine 506 is configured to command a plurality of pumps for a primary infusion and a secondary infusion, sometimes known in the art as “piggybacking.” For example, control engine 506 can command a first pump to administer a first medication, solution, or other infusate to a patient. Control engine 506 can further command a second pump to administer a second medication, solution, or other infusate along the same line, tubing, or cannula to the patient such that the second infusate is piggybacked with the first infusate. In embodiments, control engine 506 can control the flow of the first infusate with the first pump. In embodiments, control engine 506 can control the flow of the second infusate with the second pump. In embodiments, control engine 506 can control both the first infusate and the second infusate with either the first pump or the second pump. In embodiments, control engine 506 can stop the main infusion of the first infusate and allow the second infusate to be infused. After the piggyback process is finished, control engine 506 can command the first pump to restart infusion of the first infusate.

For example, while a patient is receiving an infusion of fluids, a bag of antibiotics can be hung and delivered to the patient in place of the fluids until the bag is empty, then the fluid delivery resumes. This is very similar to “flush” on a syringe pump. Additionally, this type of configuration can be used for the “mixing of drugs.” For example, a primary pump can deliver D5 W (5% dextrose in water), and subsequent pumps can introduce drugs at a rate that it is mixed to the correct ratio. This could result in a “custom drug mix that is 75% D5 W, 10% Propofol, 10% Remifentanil, and 5% Ketamine, which is a common combination of drugs for longer duration surgeries.

In an embodiment, control engine 506 is configured to command a plurality of pumps for a takeover mode. For example, control engine 506 can command a first pump to administer a first medication, solution, or other infusate to a patient. Control engine 506 can further command a second pump to take over for the first pump and administer a second medication, solution, or other infusate to a patient.

Control engine 506 can further command two or more pumps to each deliver infusate at the same time to achieve a combination therapeutic effect. In an embodiment, an infusion comprising two drugs in separate syringes can be managed as directly proportional as a single infusion or can be managed disproportionately, intentionally, with respect to the other. For example, in an anesthesia infusion, a first drug comprising a paralytic agent designed to keep the patient from moving can be delivered at the same time as a second drug comprising an anesthetic agent to keep the patient from feeling pain. In embodiments, the first drug and second drug can be administered with respect to each other.

In embodiments, control engine 506 configuration is further useful for delivering critical drugs that cannot or should not be stopped in treating a patient. For example, as one pump is running out, another pump can start. In such an operably coupled system, the handoff can be verified by a corresponding patient parameter.

Control engine 506 is further configured to establish timely control for efficacy of therapy. In other words, control engine 506 can control therapies in a time-sensitive manner to ensure the efficacy of the drugs being administered. For example, some drugs have very short half-lives or windows of action. Control engine 506 can effectively guarantee, or at least provide a relatively high degree of confidence, that the drugs (including any interacting medications or infusions) will be administered to be effective according to that half-life, on plan, on schedule, and in real time. As such, the effect of those drugs can be advantageously applied to the patient when desired or when most effective.

Messaging engine 508 is configured to execute the function or set of functions of message handling for the one or more infusion pumps. In an embodiment, messaging engine 508 can issue messages to one or more infusion pumps. In an embodiment, messaging engine 508 can receive messages from the one or more infusion pumps. In an embodiment, messaging engine 508 can receive a message from a first pump that is intended for a second pump. Once the message is received, messaging engine 508 can forward the message received from the first pump to the second pump. In another example, messaging engine 508 can receive a message from a first pump that is intended for all other communicatively coupled pumps. Once the message is received, messaging engine 508 can forward the message received from the first pump to all other such coupled pumps. In embodiments, messaging engine 508 is configured to evaluate any messages received to determine whether any action should be taken, such as forwarding to other pumps. In other embodiments, messaging engine 508 can forward messages to control engine 506, which in turn, can make operational control decisions subject to, in some embodiments, pre-defined or user-defined and real-time criteria or limits.

In embodiments, messaging engine 508 is configured to forward messages in either real-time or non-real time to an outside system, such as an HIS and/or EMR as aforementioned. For example, messaging engine 508 can utilize non-real time data packeting to relay messages to an HIS or EMR. In another embodiment, such as a gas anesthesia system with real-time coordinated IV infusions, messaging engine 508 can utilize real-time data packeting to provide delivery of the messages related to the event as closely as possible in time to the event. As such, messaging engine 508 comprises message latency management to guarantee response time and performance, and further addresses the problem of latency that can creep in with traditional systems. In embodiments, messaging engine 508 can relay messages received by an outside system. In other embodiments, messaging engine 508 can make determinations of which messages to send, including, for example, operational pumps, pumps in a standby mode, or off-line pumps. By way of the embedded server, the pump that is being programmed can activate the display of a pump in standby mode or an off-line pump to make use of that pump's display hardware. For example, the user can “swipe” certain data to the other pump such that the user can view more of the programming parameters at the same time, rather than be limited to solely the display of the operational pump.

Aggregation engine 510 is configured to execute the function or set of functions of aggregating data related to the operation of the one or more infusion pumps. In an embodiment, aggregation engine 510 comprises functions related to storing of the data. For example, aggregation engine 510 can accumulate events or data related to events on a pump. In an embodiment, aggregation engine 510 can comprise a persistent log or transaction record. Such a log or transaction record allows persistence and provides reliable delivery to an HIS or EMR for record keeping. Although not specifically illustrated herein, it is also to be appreciated and understood that in an embodiment, embedded server 500—if acting as a bridge, for example, between a low power communications network and a HIS or EMR—could provide for data persistence, thereby lowering energy requirements of infusion pumps and/or related medical devices operating on the lower power communications network.

Further, aggregation engine 510 comprises functions or algorithms related to evaluating the data. For example, maximums, minimums, averages, standard deviations, and other statistical calculations can be made by aggregation engine 510. Aggregation engine 510 is therefore configured to aggregate, summarize, or otherwise manage or evaluate the data related to the operation of the one or more infusion pumps. In an embodiment, aggregation engine 510 facilitates reducing latency for the real-time system. Embodiments of server 500 provide control of both timing and latency, so that aggregation engine 510 can timely accomplish the desired therapy.

Networking engine 512 is configured to execute the function or set of functions of network access to the one or more infusion pumps. As such, networking engine 512 can comprise software and hardware interfaces between embedded server 500 and the one or more infusion pumps. Networking engine 512 provides standardized networking functions such as message forwarding, connecting, and disconnecting. In embodiments, networking engine 512 can implement any suitable protocol layers over the network. Networking engine 512 can issue network IDs for any of the infusion pumps and itself, if needed. Networking engine 512 can therefore manage the addressing of the devices on the network.

In embodiments, any of control engine 506, messaging engine 508, aggregation engine 510, and networking engine 512 can utilize any of the respective other engines to execute the set of functions or roles described herein with respect to a particular engine. For example, control engine 506 can utilize messaging engine 508 to transmit commands in the form of messages to the one or more infusion pumps. In another example, processor 502, and particularly, control engine 506, messaging engine 508, and/or aggregation engine 510 can each utilize networking engine 512 for the interaction via the network with the one or more infusion pumps. In another example, messaging engine 508 can utilize aggregation engine 510 to aggregate, summarize, or otherwise manage or evaluate the messages received from the one or more infusion pumps.

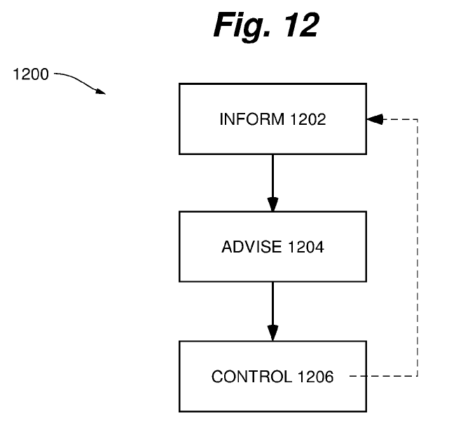

Likewise, any of control engine 506, messaging engine 508, aggregation engine 510, and networking engine 512 can utilize memory 504 to store or save data related to a respective engine. For example, control engine 506 can store control algorithms, to-be-issued control commands, or issued control commands in memory 504. In another example, messaging engine 508 can store received, transmitted, or to-be transmitted messages in memory 504. In another example, aggregation engine 510 can store evaluation algorithms, raw data, and aggregated data in memory 504. In another example, one of the aforementioned algorithms, raw data, or aggregated data can be the pharmacokinetics (PK) curves for that specific patient, provided that there is some feedback to verify the outcome, as described below with respect to FIGS. 11-14.

In another example, embodiments can calculate the end resultant drug concentration based on total flow rate for all pumps and the partial contribution to total flow rate for each pump/drug. In further embodiments, systems can calculate and display the pharmacokinetic curves for the drugs/drug combinations that are being administered, similar to the pharmacokinetic (PK) models used for target-controlled infusion (TCI). In another example, the data can be considered “verified” if the medical professional is adjusting the medication administration based on the hospital's standard procedures. In another example, networking engine 512 can store in memory 504 network addresses or hardware information for each of the connected (or potentially connected) infusion pumps.

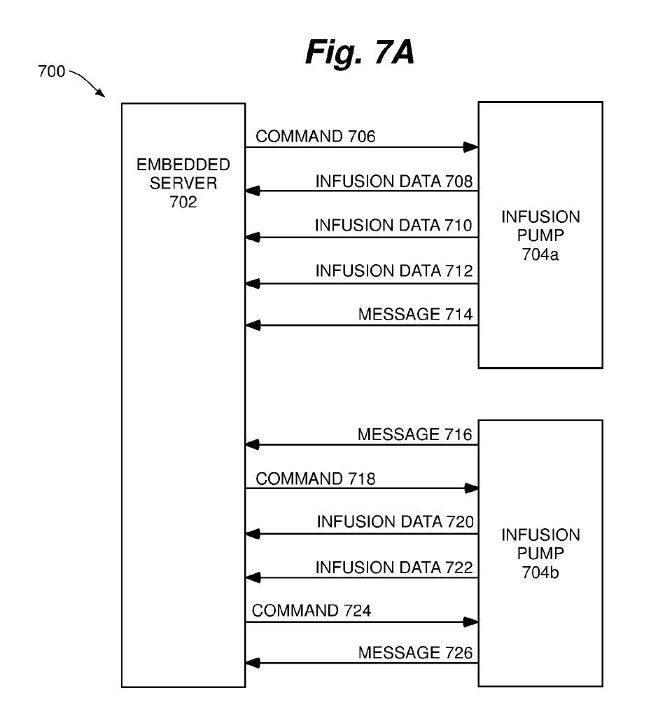

Referring to FIG. 6, a block diagram of an example of a real-time embedded server system 600 and an external hospital information system 606 is depicted, according to an embodiment. As depicted, embedded server system 600 generally comprises embedded server 500, a first infusion pump 604a, and a second infusion pump 604b. Hospital information system 606 is external to embedded server system 600 and can be operably coupled to embedded server 500. Further, while FIG. 6 is depicted with two infusion pumps 604a and 604b, system 600 can comprise one or a plurality of infusion pumps interfacing to embedded server 500. In an embodiment, infusion pumps 604a and 604b can be similar in selected features, whether in part or entirely, to infusion pump 210 described above.

For example, and with reference to both FIG. 5 and FIG. 6, embedded server 500 can utilize control engine 506 and networking engine 512 to issue a pump command 608 to infusion pump 604a. Pump command 608 can comprise an infusion operation or protocol. Infusion pump 604a can receive command 608 and transmit a confirmation 610 back to embedded server 500, that command 608 was received by infusion pump 604a. Confirmation 610 can be received by networking engine 512 of server 500. Subsequently, or in parallel, infusion pump 604a can implement the infusion operation commanded by command 608.

In another example, embedded server 500 can utilize messaging engine 508 and networking engine 512 to issue a message 612 to infusion pump 604b. Message 612 can be, for example, a request to provide data related to the operation of infusion pump 604b. In response, data 614 can be transmitted back to embedded server 500. Data 614 can be received by networking engine 512 of server 500. In another embodiment (not shown), message 612 can be a message from infusion pump 604a. In another embodiment (not explicitly shown), message 612 can be a message received by an outside system such as hospital information system 606 to be transmitted by embedded server 500 to one or more of the infusion pumps (for example, coupled infusion pump 604b.)

In an embodiment, although not explicitly shown in the drawings, networking engine 512 is configured to interface to hospital information system 606. Any of pump command 608 transmitted to infusion pump 604a or confirmation 610 received from infusion pump 604a can be transmitted to hospital information system 606. Likewise, any of message 612 transmitted to infusion pump 604b or data 614 received from infusion pump 604b can be transmitted to hospital information system 606. In another embodiment, aggregation engine 510 can be utilized to aggregate any of command 608, confirmation 610, message 612, or data 614 to provide a more complete communication or status to hospital information system 606.

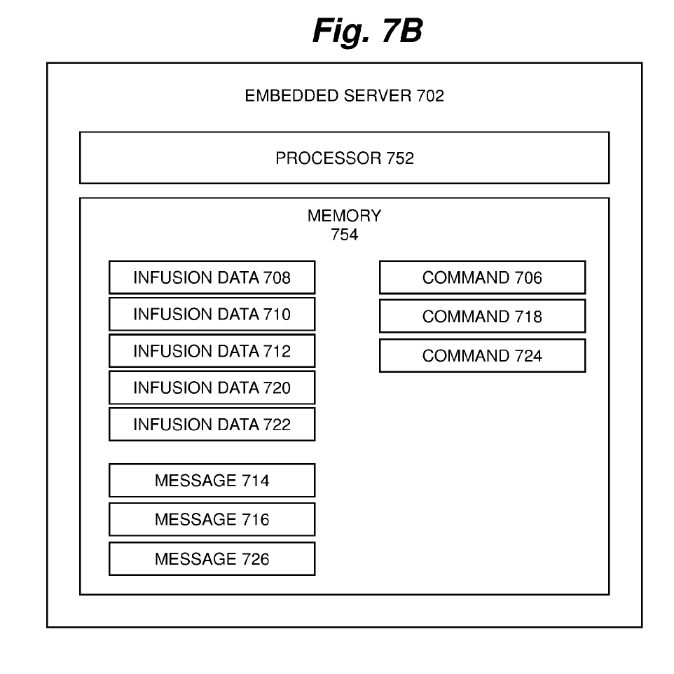

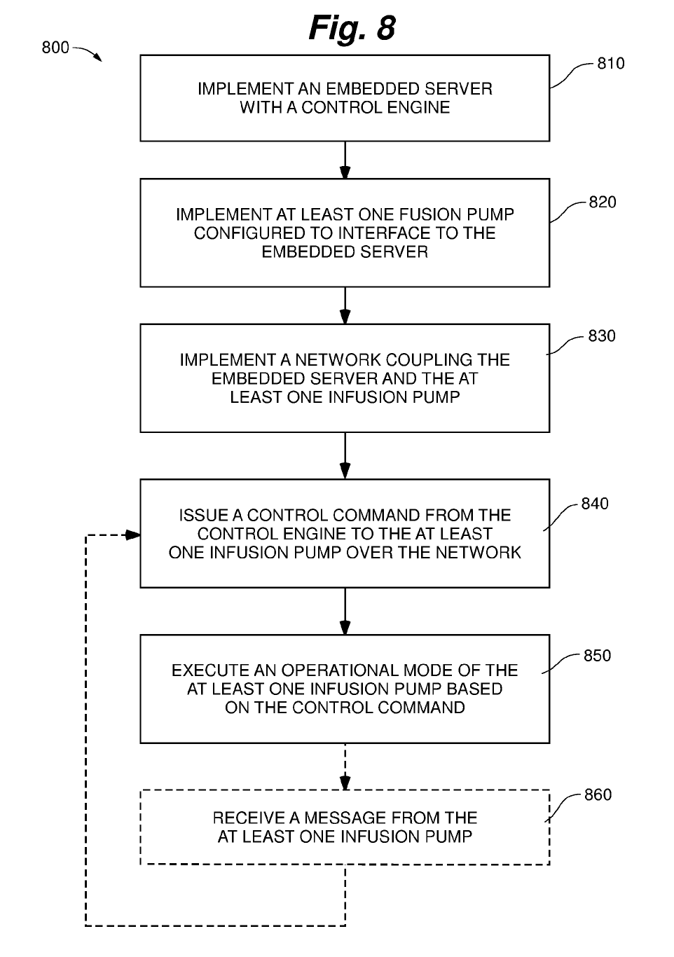

Referring to FIG. 7A, a block diagram of an example of a real-time embedded server system 700 including messaging traffic between an embedded server 702 and infusion pumps 704a and 704b is depicted, according to an embodiment. In embodiments, embedded server 702 can be similar in selected features, whether in part or entirely, to embedded server 215, embedded server 302, embedded server 402, or embedded server 500. Likewise, infusion pumps 704a and 704b can be similar in selected features, whether in part or entirely, to infusion pump 210 described above. The sequencing time-wise in the example of FIG. 7A is to be read from top to bottom, although the subject matter hereof—as described by example or otherwise contemplated herein—is not necessarily limited to such a chronological sequence.

In operation, embedded server 702 can issue a command 706 to infusion pump 704a. In an embodiment, command 706 comprises a pump operation command and status request. For example, the status request can comprise a request for infusion data every 10 seconds. In response, infusion pump 704a begins pump operation and subsequently transmits back to embedded server 702 infusion data 708 at 10 seconds of pump operation, infusion data 710 at 20 seconds of pump operation, and infusion data 712 at 30 seconds of pump operation. A message 714 is then transmitted from infusion pump 704a to embedded server 702.

Referring to operation between embedded server 702 and infusion pump 704b, message 716 can initiate messaging with embedded server 702. Embedded server 702 can evaluate message 716 and subsequently issue command 718. In an embodiment, command 718 comprises a pump operation command. In response, infusion pump 704b begins pump operation. In embodiments, embedded server does not request a status in command 718, as embedded server 702 has made a determination that infusion pump 704b is preconfigured to transmit data during operation. Accordingly, infusion data 720 and infusion data 722 are transmitted from infusion pump 704b to embedded server 702. In response, embedded server 702 is configured to evaluate infusion data 720 and infusion data 722 and issue command 724 that updates the operation of infusion pump 704b. For example, if infusion data 720 and infusion data 722 indicate that the patient is not receiving the initially-commanded medication at an appropriate rate by command 718, embedded server 702 can update the rate in command 724. Infusion pump 704b subsequently updates its rate of infusion and issues message 726 back to embedded server 702. Such examples of operation of system 700 illustrate the real-time control provided by embedded server 702.

In embodiments, operation of embedded server 702 with infusion pump 704a and infusion pump 704b can be concurrent or in series. While FIG. 7A is depicted with two infusion pumps 704a and 704b, system 700 can comprise one or a plurality of infusion pumps interfacing to embedded server 702.

Referring to FIG. 7B, a block diagram of the example of real-time embedded server 702 of FIG. 7A is depicted which illustrates the storage of the aforementioned messaging traffic, according to an embodiment. In an embodiment, embedded server 702 generally comprises processor 752 and memory 754. Processor 752 can be similar in selected features, whether in part or entirely, to processor 502. Likewise, memory 754 can be similar in selected features, whether in part or entirely, to memory 504. In an embodiment, memory 754 can comprise volatile memory for operation of the processor and a separate database for storing records related to system 700.

Memory 754 can be configured to store the messaging traffic, commands, or data transferred between embedded server 702 and infusion pumps 704a and 704b. For example, infusion data 708, infusion data 710, infusion data 712, infusion data 720, and infusion data 722 can be stored, saved, or otherwise recorded in memory 754 as individual records. Likewise, message 714, message 716, and message 726 can be stored, saved, or otherwise recorded in memory 754 as individual records. Command 706, command 718, and command 724 can be stored, saved, or otherwise recorded in memory 754 as individual records.

As shown in FIG. 7B, the infusion records are stored together, the message records are stored together, and the command records are stored together. In embodiments, the infusion records, the message records, and the command records can be stored such that they are intermixed within memory 754. In such an embodiment, memory 754 can be separately configured to store pointers to the respective records so that groupings or categories can be efficiently retrieved. In other embodiments, summary or aggregate data that summarizes or aggregates the infusion data, message data, and command data is stored in memory 754 in addition to the individual records. In another embodiment, only the summarized or aggregated data is stored in memory 754.

Although not explicitly illustrated in FIG. 7B, but referring analogously to the components of FIG. 5, in an embodiment of embedded server 702 comprising a control engine, messaging engine, aggregation engine, and networking engine, any of these engines can access the records stored in memory 754. In other embodiments, memory 754 can be restricted such that only one or some of the engines can access the records stored in memory 754. Such permission-based access allows for secure and confidential storage of infusion pump-server data.

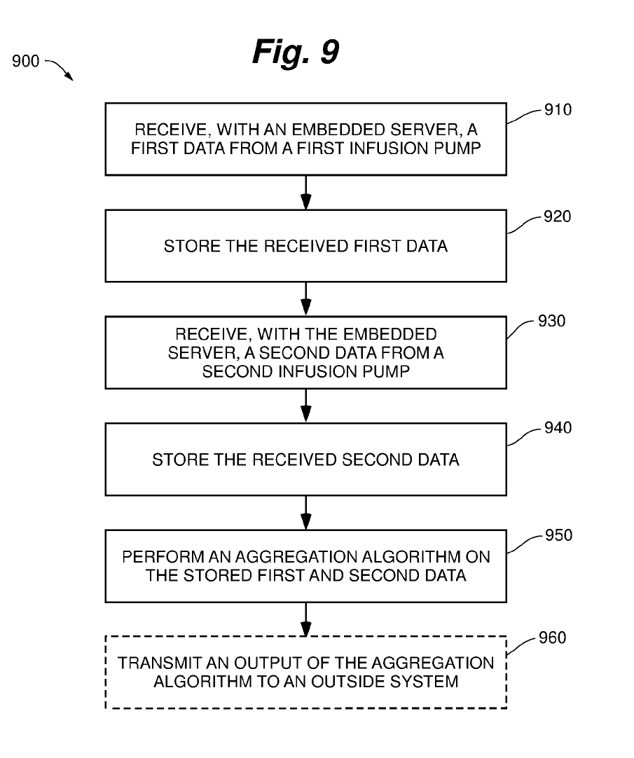

Referring to FIG. 8, a flowchart of an example of a method 800 of controlling a plurality of infusion pumps with a real-time embedded server is depicted, according to an embodiment.

At 810, a real-time embedded server having a control engine is implemented. As described analogously with respect to FIG. 5, the control engine is configured to issue control commands to at least one infusion pump, wherein the control commands are related to the operation of the at least one infusion pump.

At 820, at least one infusion pump is implemented. The at least one infusion pump is configured to interface to the embedded server of 810.

At 830, a network coupling the embedded server of 810 and at least one infusion pump of 820 is implemented. For example, the infusion pump can be similar in selected features, whether in part or entirely, to infusion pump 210 as described with respect to FIG. 2. In embodiments, a plurality of infusion pumps can be implemented. In an embodiment, multiple embedded servers can be implemented via the same network. For example, a first server can interface to a first infusion pump on the network, and a second server can interface to a second and third infusion pump on the network. In such embodiments, the first and second servers can interface with each other to control first, second, and third infusion pumps.

At 840, a control command can be issued from the control engine of 810 to the at least one infusion pump of 820. The control command is transmitted from the embedded server to the at least one infusion pump via the network of 830.

At 850, the control command of 840 is received by the at least one infusion pump of 820 and an operational mode of the at least one infusion pump is executed based on the received control command. For example, the control command can instruct the at least one pump to administer a first medication or solution to a patient.

Optionally at 860, a message is received from the at least one infusion pump of 820. The message received from the at least one infusion pump at 860 can comprise a confirmation of the control command of 840. In embodiments, the embedded server of 810 can cancel or otherwise inhibit the control command previously issued unless the confirmation is received. In another embodiment, the message received from the at least one infusion pump can comprise a status or operational data to create feedback for a system of method 800. In such embodiments, the status or operational data can be incorporated into an additional control command issued at 840. For example, as illustrated in FIG. 8, from 860, the method optionally returns to 840, where another control command can be issued.

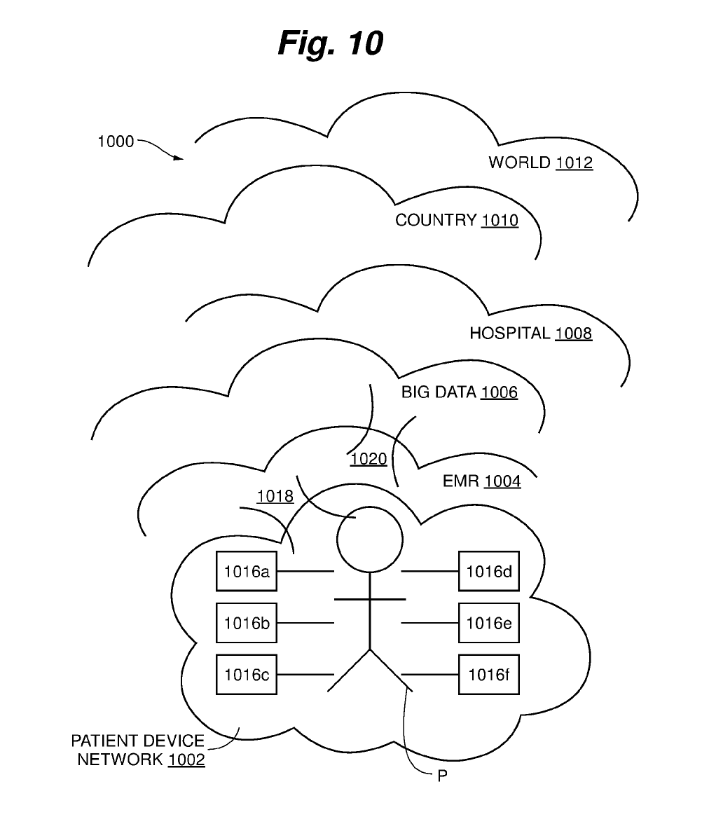

Referring to FIG. 9, a flowchart of an example of a method 900 of aggregating data related to a plurality of infusion pumps with a real-time embedded server is depicted, according to an embodiment. In an embodiment, and referring analogously again to FIG. 5, method 900 can be implemented, for example, by portions or all of control engine 506, messaging engine 508, aggregation engine 510, and networking engine 512.

At 910, a first data is received with an embedded server from a first infusion pump. In an embodiment, the first data comprises a message. In another embodiment, the first data comprises data related to the operation of the first infusion pump. In other embodiments, the first data can comprise network status data. In other embodiments, the first data can comprise other data related to the first infusion pump or any coupled components interfacing to the first infusion pump.

At 920, the first data is stored by the embedded server of 910. In an embodiment, the first data can be stored in electrically coupled memory of the embedded server of 910. In another embodiment, the first data can be stored in a database operably coupled to the embedded server.

At 930, a second data is received with the embedded server from a second infusion pump. In an embodiment, the second data comprises a message. In embodiments, the second data can comprise a message for the first infusion pump. In another embodiment, the second data comprises data related to the operation of the second infusion pump. In other embodiments, the second data can comprise network status data. In other embodiments, the second data can comprise other data related to the second infusion pump or any coupled components interfacing to the second infusion pump.

At 940, the second data is stored by the embedded server of 930. In an embodiment, the second data can be stored in electrically coupled memory of the embedded server of 930. In another embodiment, the second data can be stored in a database operably coupled to the embedded server.

At 950, an aggregation algorithm is performed on the stored first data of 920 and the stored second data of 940. The aggregation algorithm comprises functions or algorithms related to evaluating the stored data. For example, maximums, minimums, averages, standard deviations, and other statistical calculations can be made by the aggregation algorithm. The aggregation algorithm is therefore configured to aggregate, summarize, or otherwise manage or evaluate the stored data.

Optionally, at 960, an output of the aggregation algorithm performed at 950 can be transmitted to an outside system. For example, a status of the operating first infusion pump of 910 and second infusion pump of 930 can be provided to a hospital information system. In an embodiment, and referring analogously to FIGS. 5 and 6, a networking engine is configured to interface to a hospital information system.

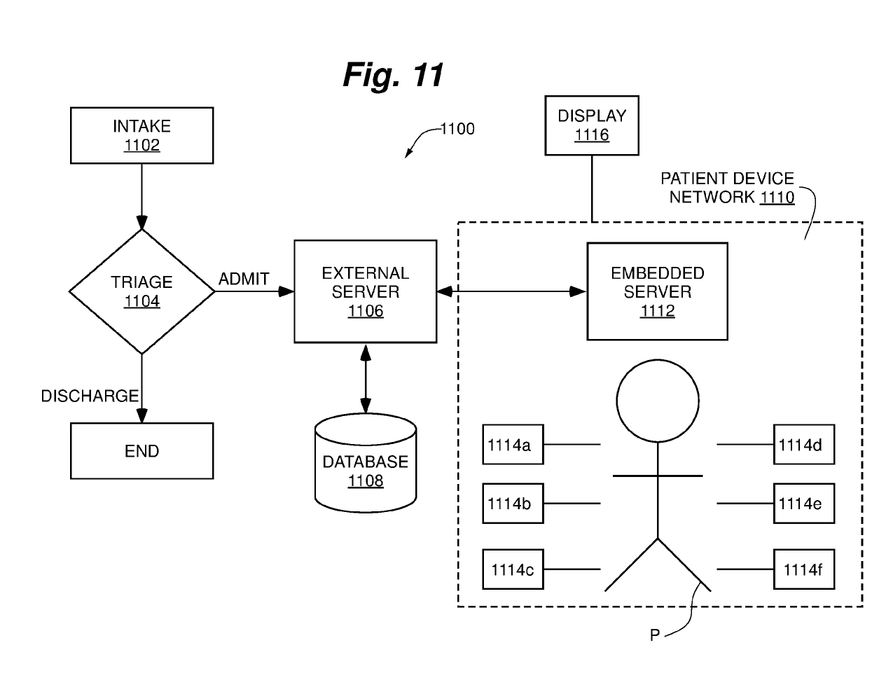

In an embodiment, referring to FIG. 10, a block diagram of an example of a system 1000 of patient care networks is depicted. System 1000 is depicted in FIG. 10 in a so-called “cloud” computer/medical device architecture (although “cloud” components, systems, and architectures are not necessarily required in various embodiments of systems of patient care networks as described or otherwise contemplated herein) and generally comprises a patient-device network 1002, EMR network 1004, big data network 1006, hospital network 1008, country-level network 1010, and world-level network 1012. Each network 1002-1012 can comprise one or more computing devices communicatively coupled to each other or to portions of the other networks. For example, one or more servers can be operably coupled to the Internet or a particular intranet to represent a particular network. Each network 1002-1012 can further comprise one or more databases configured to store data respective to that particular network. FIG. 10 depicts networks 1002-1012 as a hierarchy for ease of explanation, but networks 1002-1012 can be implemented in nonhierarchical architectures as well. Of course, system 1000 can be implemented with additional or fewer networks, or different networks than depicted. Moreover, although data has been defined in groups or levels, any suitable group, groups, level, or levels can be defined or utilized. For example, big data network 1006 can refer to that level or any other suitable level of data.

Patient-device network 1002 can comprise a patient P operably coupled to a plurality of medical devices 1016a-1016f As will be described further below, medical devices 1016a-1016f can each comprise a device for providing a therapy or medication to patient P. Further, medical devices 1016a-1016f can each comprise a device for reading a status or characteristic of the patient, such as a sensor.

EMR network 1004 can comprise one or more computing devices and/or databases configured to manipulate and store electronic medical record data. Big data network 1006 can comprise one or more computing devices and/or databases configured to manipulate and store aggregated device-level and EMR data. Hospital network 1008 can comprise one or more computing devices and/or databases configured to manipulate and store hospital-level data. Country network 1010 can comprise one or more computing devices and/or databases configured to manipulate and store country-level data. World network 1012 can comprise one or more computing devices and/or databases configured to manipulate and store world-level data. Any of the data stored or manipulated at a respective network level can be aggregated from other networks, or stored or manipulated individually, depending on the data and the application.

Each of the subsequent networks can access the immediately-adjacent network. As illustrated, access to patient-device network 1002 by EMR network 1004 can be implemented by a real-time embedded server 1018. Embedded server 1018 can receive data from EMR network 1004 for transmission to patient-device network 1002. Embedded server 1018 can further receive data from patient-device network 1002 for transmission to EMR network 1004.

In another example, big data 1006 can access EMR network 1004 data over a suitable interface or bridge 1020. Bridge 1020 can comprise, for example, an electronic medical record server or system, such as a CERNER or EPIC system. Bridge 1020 can therefore receive data from big data 1006 for transmission to EMR 1004. Similarly, bridge 1020 can receive data from EMR 1004 for transmission to a collection of big data 1006. Likewise, hospital network 1008 can access big data 1006 over a similarly suitable interface or bridge, and so on. Other bridges are not depicted in FIG. 10 for simplicity, but one skilled in the art will readily understand the interface concepts provided by the examples of embedded server 1018 and bridge 1020 as being applicable, analogously, to other components of system 1000. In other embodiments, networks 1002-1012 can access data of networks that are not immediately adjacent each other (literally or figuratively), for example, by databases or interfaces provided by the respective network.

As will be described further below, once patient-device network 1002 includes access to other levels or networks (for example, by embedded server 1018 and bridge 1020, etc.), myriad therapies can be implemented. For example, effectiveness or efficiency data can be compared across networks for real-time updating of infusion parameters used in patient-device network 1002. In other embodiments, pattern data available across or between networks can be utilized for real-time updating of infusion parameters used in patient-device network 1002. Different hierarchical levels also can provide different granularity, depth, or detail of data. Data at a hospital level (for example, from hospital network 1008) can differ from data at a world level (for example, at world network 1012). Particular trends or patterns in the data can be determined for a particular, therapy, or patient, or classification of patients as needed or desired. For example, embodiments can track all delivery of a particular drug at any level in the hierarchy to assess organization risk.

In another example, varying levels of business rule constraints, such as flow limits, drug limits, various settings (e.g. safety limits), alarm sound types, etc. can be coordinated or set according to the access to other levels or networks. A multinational hospital system could require the same sound settings across all hospitals, for example.

Referring to FIG. 11, a block diagram of a decentralized embedded real-time server system 1100 is depicted, according to an embodiment. Generally, system 1100 comprises an external server 1106, such as a Smiths Medical PHARMGUARD server system, a database 1108, and a patient-device network 1110.

External server 1106 can comprise a computing device configured to manipulate and store data related to infusion therapies of patient-device network 1110 and other similar networks. In an embodiment, external server 1106 is communicatively coupled to database 1108. Database 1108 is configured to store data related to infusion therapies. For example, referring again to FIG. 10, external server 1106 and database 1108 can comprise a portion of EMR network 1004, or big data 1006, or hospital 1008, as appropriate. In most embodiments, external server 1106 and database 1108 comprise an architecture one level “above” patient-device network 1002 of FIG. 10.

Patient-device network 1110 of FIG. 11 can be substantially similar to patient-device network 1002 as described in FIG. 10. As depicted in FIG. 11, patient-device network 1110 is communicatively coupled to external server 1106. In an embodiment, patient-device network 1110 comprises an embedded server 1112 and a plurality of devices 1114a-1114f. Embedded server 1112 is decentralized from any hospital network, which enables real-time, dynamic, and patient-specific informed control of devices 1114a-1114f Embedded server 1112 can comprise equations, algorithms, or other processor-based instructions for manipulating any of the inputs or outputs from external server 1106 or devices 1114a-1114f In embodiments, embedded server 1112 can comprise a medical device, a plurality of medical devices, a computer, a tablet computer, a portable phone, a rack, or a module adapted to be plugged into a system component.

In one example, devices 1114a-1114c comprise inputs to embedded server 1112, while devices 1114d-1114f comprise outputs from embedded server 1112. Devices 1114a-1114c are each a sensor or monitor for patient P. For example, devices 1114a-1114c can respectively comprise a respiratory sensor, a blood pressure sensor, and a heart rate sensor. Each of input devices 1114a-1114c are therefore configured to sense data or status from patient P. Devices 1114d-1114f are each a delivery device for patient P. For example, devices 1114d-1114f can each respectively comprise an infusion pump. Embedded server 1112 is therefore configured to receive the data from input devices 1114a-1114c and respectively program devices 1114d-1114f based on the input from input devices 1114a-1114c and any other programming commands or input from external server 1106. Feedback mechanisms other than input devices 1114a-1114c are also considered.

In an embodiment, downloadable software modules can add additional features, functionality, or algorithms to systems described herein. A downloadable module can register to a central “rules engine” so that the module functions are accessed according to defined rules. For example, when a particular therapy is used, a command or sequence of commands to manage multiple drugs with a special algorithm with automatic delivery changes based on vital signs can be executed.

System 1100 can further comprise display 1116. In an embodiment as shown in FIG. 11, display 1116 can be external to patient-device network 1110. In other embodiments, display 1116 can be integrated into patient-device network 1110. Display 1116 can be utilized to show or illustrated individual device data or aggregated data, including multi-drug treatments such as TCI.

Although not specifically illustrated herein, it is also to be appreciated and understood that embedded server 1112 could advantageously be configured or designed to combine information from multiple analogous or related sensors and/or monitors for patient P into a single and more accurate or dependable data stream.

In other embodiments, a myriad of communication schemes can be achieved by the architecture and interfaces according to real-time server system 1100. For example, peer-to-peer communication can be implemented between any of devices 1114a-1114f, or between any of devices 1114a-1114f and an external device (not shown in FIG. 11). In other embodiments, adhoc wireless connectivity can be implemented, wherein external server 1106 is designated as a peer, and another device is designated as the master, such as an individual device 1114a or embedded server 1112.

Other elements of system 1100 shown as process steps interfacing to the aforementioned external server 1106 and patient-device network 1110 are described below.

In operation, intake 1102 on patient P is conducted. In an embodiment, intake 1102 comprises gathering information from patient P. In an embodiment, intake 1102 can be conducted manually by a nurse or other practitioner. In other embodiments, an automated intake 1102 can be conducted; for example, by external server 1106.

At 1104, triage is conducted to determine patient P's condition from data or information received at intake 1102. In an embodiment, triage 1104 comprises a nurse or other practitioner analyzing the intake 1102 data or information about patient P. In another embodiment, an automated triage 1104 is conducted on data or information from intake 1102 by a computing device, such as external server 1106.

From triage 1104, patient P is either admitted to or discharged from a medical facility. If patient P is admitted, then for example the aforementioned ICD codes as well as data or information from intake 1102 can be inputted to external server 1106. If patient P is discharged, the system proceeds to an end state.

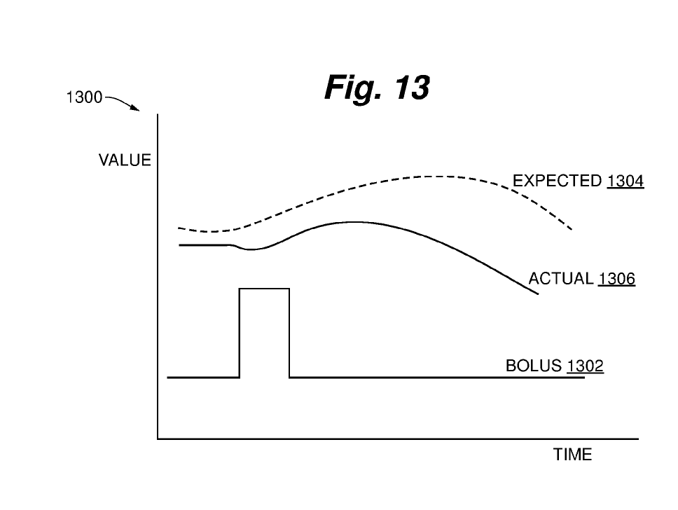

Returning again to the case where patient P is admitted from triage 1104, one or more patient-specific therapies based on an “inform-advise-control” scheme can be generated by system 1100. Referring to FIG. 12 (with continued reference also to FIG. 11), a flowchart of an inform-advise-control medical device scheme 1200 is depicted, according to an embodiment. For example, an initial infusion can be administered to patient P. The initial infusion can be programmed or stipulated according to intake 1102 data or information and external server 1106 data (stored on database 1108), for a patient having those particular characteristics. Patient P's specific reaction can be monitored by input devices 1114a-1114c. Therefore, specific circumstances or reactions of particular patients can be compared to expected circumstances or reactions from data stored on database 1108. In an embodiment, when patient P is admitted, embedded server 1112 can be associated with the patient and the associated devices 1114 can be associated per their use, connection point, or other functionality or data element, wherein advanced algorithms can be employed to optimize patient care.

In an “inform” step 1202, data from input devices 1114a-1114c can be gathered by embedded server 1112. In an embodiment, embedded server 1112 can aggregate data from input devices 1114a-1114c. This data can be transmitted by embedded server 1112 to external server 1106 and presented by external server 1106 to patient P or nurse or other practitioner. In another embodiment, the data can be presented on one of the devices 1114a-1114f Relative success or failure indications can be provided based on the status data of 1114a-1114c. Further, relative warning or “soft limit” indications can be further provided.

In an “advise” step 1204, data from input devices 1114a-1114c of FIG. 11 can be incorporated by external server 1106 or embedded server 1112 to advise a subsequent action. Data from database 1108, communicated via an interface between external server 1106 and database 1108, can further be used for comparison, contrast, or advising. In an embodiment, data in database 1108 can comprise data from other inputs such as ventilator data, extracorporeal membrane oxygenation (ECMO) data, laboratory data, or any of the hierarchy level described relative to the networks of FIG. 10, such as device-level data, EMR data, big data, hospital-level data, country-level data, and world-level data. A subsequent action can be to recommend or advise a change to the medication being administered. In such a case, the data from input devices 1114a-1114c might indicate a reaction by patient P that is outside of expected boundaries. In another example, subsequent action can be to further monitor patient P during medication administration and so advise the nurse or other practitioner.

In an optional “control” step 1206, devices 1114d-1114f of FIG. 11 can be reprogrammed or otherwise commanded to re-administer or change the infusions being given, based on the analysis of data conducted in the “advise” step 1204. In an embodiment, the reprogramming recommendation is presented to a user for acceptance in the “advise step” 1204, or by another suitable technique, before it is administered to patient P. As depicted, from control 1206, “inform-advise-control” scheme 1200 can optionally proceed back to inform 1202, such that the process can be iterated. Iterations can occur at any desired frequency; for example, after a certain period of time into an infusion protocol. In another embodiment, iterations can occur at an independent time or mark, such as every hour or every 15 minutes. Dynamic control of infusions, using “inform-advise-control” scheme 1200, can therefore be achieved.

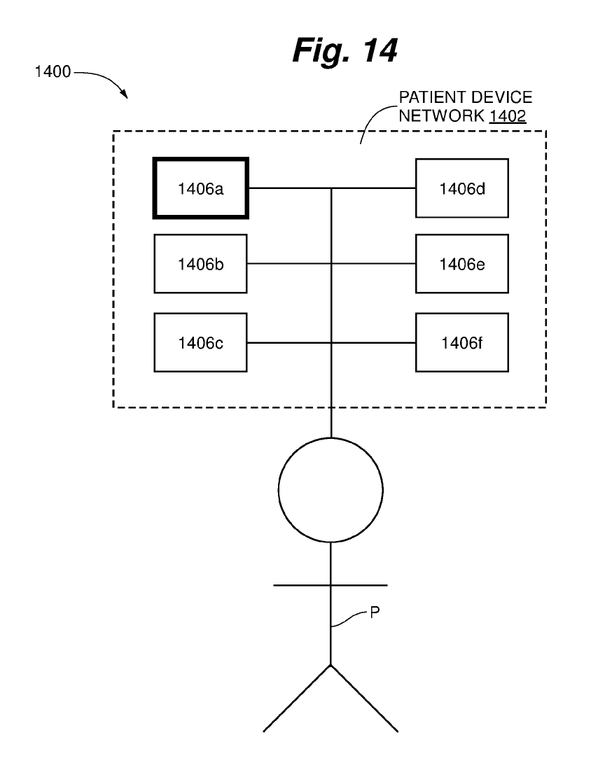

Referring to FIG. 13, a graph 1300 of a medication administration with respect to time in combination with expected and actual patient characteristic values resulting from the medication is depicted, according to an embodiment. Graph 1300 depicts time on the x-axis and relative values on the y-axis. As shown, a bolus 1302 is delivered to the patient at a given time. An expected value 1304 for a particular patient characteristic in relation to bolus 1302 is shown (and can be stored as data in database 1108 of FIG. 11, for example). Subsequently, as measured as part of “inform-advise-control” scheme 1200 of FIG. 12 described above, an actual value 1306 of the particular patient characteristic can be measured. If actual value 1306 of the particular patient characteristic differs from expected value 1304, a remedial action can be taken for the particular patient being medicated. As shown in the example of FIG. 13, actual value 1306 differs from expected value 1304 in both shape and relative magnitude of value.

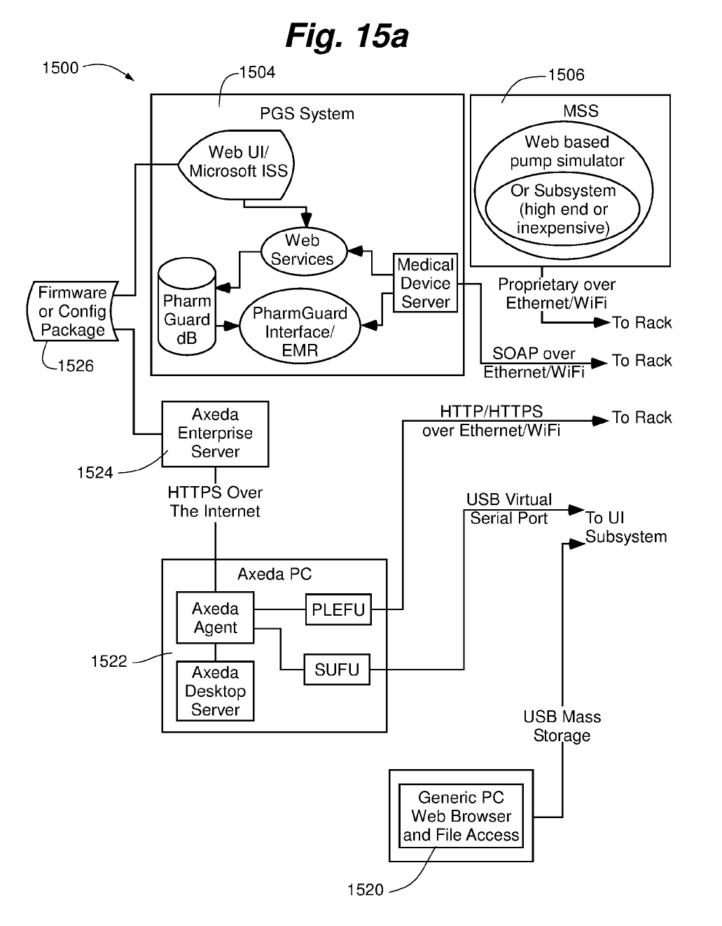

Referring to FIG. 14, a block diagram of a patient care system 1400 including at least one embedded server controlling device is depicted, according to an embodiment. In an embodiment, patient care system 1400 comprises a patient-device network 1402 for treating a patient P.

In an embodiment, patient-device network 1402 comprises a plurality of medical devices 1406a-1406f. Medical devices 1406a-1406f can comprise input or output devices as described, for example, with respect to devices 1114a-1114f in FIG. 11. For example, each medical device 1406a-1406f can comprise a sensor or monitor or delivery device for patient P.

Additionally, patient-device network 1402 includes a controlling device having the functionality of an embedded server. In the example depicted, medical device 1406a is the controlling device (represented in FIG. 14 by a bold rectangular border) and is configured to control itself and medical devices 1406b-1406f. For example, medical device 1406a can include a module similar to control module 220 of FIG. 2. Medical device 1406a is therefore configured to administer medication to patient P or sense data about patient P, as the case may be; and device 1406a can also provide, in some embodiments, and overall “inform-advise-control” scheme as aforementioned with respect to infusion pumps and other associated devices medical devices 1406b-1406f.

For example, controlling device 1406a can establish a 1-1 relationship between a therapy and a drug, such as the administration of anesthesia. In an anesthesia therapy example, one of the medical devices 1406a-1406f can be commanded to administer the anesthesia drug. In embodiments, the sensor-type medical devices can be commanded to sense characteristics related to the anesthesia therapy.

In another example, controlling device 1406a can establish a multiple relationship between drugs. For example, the relationship between fluid loading and blood pressure requires that multiple infusions be monitored in relation to blood pressure. Should a plurality of infusions be desired, controlling device 1406a can command multiple medical devices 1406b-1406f to administer a medication to patient P. However, each respective infusion can be considered and adapted to the other by controlling device 1406a so that the patient is not over-administered a total amount of fluid. In such an embodiment, a suitable one of medical devices 1406b-1406f can be commanded to monitor blood pressure in relation to the multiple infusions.

In an embodiment, a hierarchy of “controlling” devices can be negotiated between medical devices 1406a-1406f. For example, controlling device 1406a can designate another of medical devices 1406b-1406f as a temporary agent to control itself and the rest of medical devices 1406a-1406f. Control can subsequently be passed back to controlling device 1406a at a certain time or when a certain task has completed. In another embodiment, controlling device 1406a can be designated as the top or primary controlling device. Control of certain subsets of devices 1406b-1406f can be passed to other devices on patient-device network 1402. For example, medical device 1406b can be configured for control of medical devices 1406c-1406d, while medical device 1406e can be configured for control of medical device 1406f. In such an embodiment, medical device 1406b and medical device 1406e can pass status data back to top or primary controlling device 1406a. Such organizations of medical devices 1406a-1406f are described herein by way of example. One skilled in the art will readily appreciate that any number of hierarchies or communication structures can be implemented once patient-device network 1402 is established as described herein.

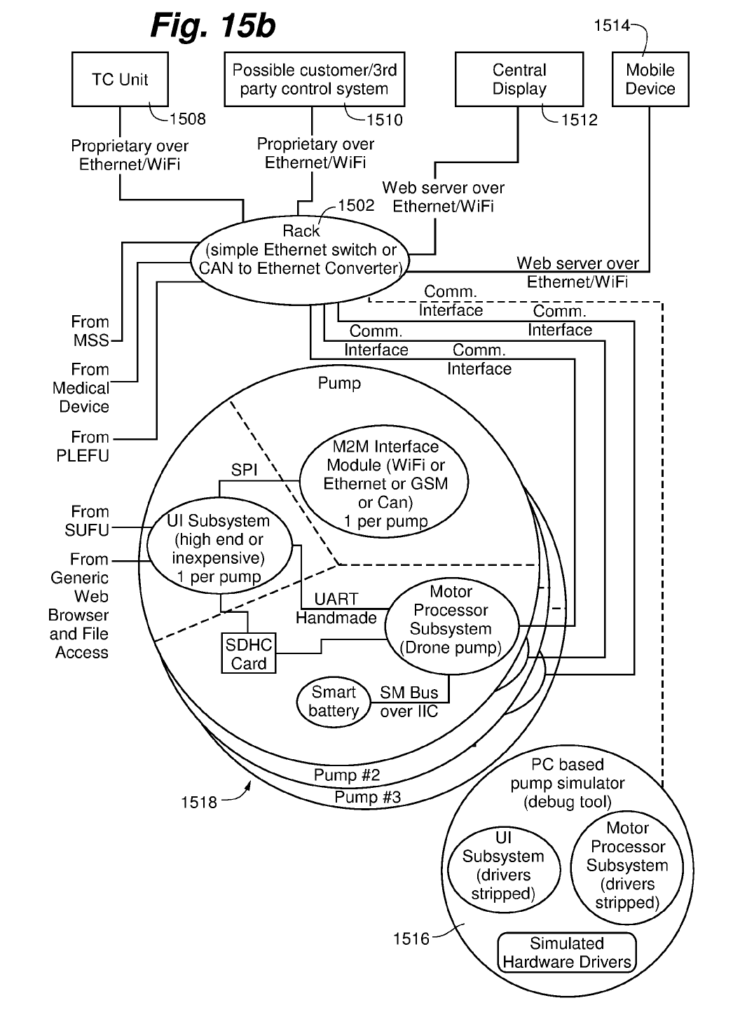

Referring to FIG. 15, a block diagram of example communication interfaces for a patient care system is depicted, according to an embodiment. A patient care system 1500 generally comprises various communications interfaces to rack 1502. Rack 1502 can comprise, for example, a simple Ethernet switch or controller area network (CAN bus) to Ethernet converter, or any other any suitable communication interface or protocol, including WiFi, Bluetooth, CAN, Ethernet, etc. hardware, firmware, software, or combinations thereof. One skilled in the art will readily appreciate that the particular interfaces or particular hardware components are described herein for example only, and any suitable network interface or protocol can be utilized, such as WiFi Adhoc, Bluetooth PAN or other suitable technology.

In an embodiment, patient care system 1500 comprises an external server 1504, such as the aforementioned Smiths Medical PHARMGUARD server (PGS) system. In an embodiment, external server 1504 is configured to communicate with rack 1502 via SOAP messaging over Ethernet/WiFi.

In an embodiment, patient care system 1500 comprises a medication safety software (MSS) system 1506. As depicted, MSS can include a web-based pump simulator and user interface (UI) subsystem. In embodiments, the web-based pump simulator can be integrated within the MSS and can use data from a currently-selected MSS configuration. MSS system 1506 is configured to communicate with rack 1502 using, in an embodiment, a proprietary Smiths Medical “CADD Solis NCS” communication protocol over Ethernet/WiFi. In an embodiment, communications can use a “reliable” sub-protocol.

In an embodiment, patient care system 1500 comprises a transmission control (TC) unit 1508. In such embodiment, TC unit 1508 is configured to communicate with rack 1502 using the aforementioned proprietary CADD Solis NCS communication protocol over Ethernet/WiFi. In an embodiment, such communications can use a “reliable” sub-protocol.

In an embodiment, patient care system 1500 comprises a possible customer/third party control system 1510. In such embodiment, customer/third party control system 1510 is configured to communicate with rack 1502 using the aforementioned proprietary CADD Solis NCS communication protocol over Ethernet/WiFi. In an embodiment, such communications can use a “reliable” sub-protocol.

In an embodiment, patient care system 1500 comprises a central display 1512. In such embodiment, rack 1502 or pumps 1518 coupled to rack 1502 can be configured to act as a web server, thereby commanding central display 1512 to provide status in a suitable pump language. In such embodiment, central display 1512 is therefore configured to communicate with rack 1502 using the aforementioned proprietary CADD Solis NCS communication protocol over Ethernet/WiFi. In an embodiment, such communications can use a “reliable” sub-protocol.

In an embodiment, patient care system 1500 comprises a handheld or mobile device 1514 such as a smartphone. In such embodiment, rack 1502 or pumps 1518 coupled to rack 1502 can be configured to act as a web server, thereby commanding handheld or mobile device 1514 to provide status in a suitable the pump language. In such embodiment, handheld or mobile device 1514 is therefore configured to communicate with rack 1502 using the aforementioned proprietary CADD Solis NCS communication protocol over Ethernet/WiFi. In an embodiment, such communications can use a “reliable” sub-protocol.

In an embodiment, patient care system 1500 comprises pump simulator 1516 such as a PC-based debug tool. In such embodiment, pump simulator 1516 can comprise a user interface (UI) subsystem having drivers removed or stripped, a motor processor subsystem having drivers removed or stripped, and simulated hardware drivers. Pump simulator 1516 is configured to communicate with rack 1502 via any suitable communication interface or protocol, including WiFi, Bluetooth, CAN, Ethernet, etc.

In an embodiment, patient care system 1500 comprises one or more pumps 1518. Each pump 1518 can include a “machine-to-machine” (M2M) interface module, a motor processor subsystem coupled to a battery, a user interface (UI) subsystem, and a secure digital high capacity (SDHC) card. In embodiments, as depicted, the motor processor subsystem can communicate to the battery along an SM bus over I2C. Further, the motor processor subsystem can communicate with the US subsystem using UART+handshaking. In embodiments, the motor processor subsystem can comprise a drone pump. The SDHC card can include one or more firmware updates, one or more tech service manuals, one or more user manuals, history log data, one or more instrumentation logs, and one or more instrumentation configuration files. SDHC card can be presented as public content as emulated mass storage over USB. In embodiments, each pump is configured to communicate with rack 1502 via any suitable communication interface or protocol, including WiFi, Bluetooth, CAN, Ethernet, etc. In an embodiment, all connections to rack 1502 are duplicated with connections direct to the pump M2M interface module.

As depicted in FIG. 15, system 1500 can further comprise a personal computer (PC) 1520. PC 1520 can include a web browser and file access subsystem. One or more pumps 1518 can communicate with PC 1520 over a USB connection. In such embodiment, each pump 1518 is configured to emulate a standard USB Mass Storage Device class. Each pump 1518 can provide full history logs as a plain ASCII file in the emulated Mass Storage Device file system. In embodiments, each pump 1518 can accept a firmware upgrade in the form of a standard S-Record file copied to the emulated Mass Storage Device.

Patient care system 1500 can further comprise an Axeda personal computer (PC) 1522. Axeda PC 1522 can be configured to communicate with rack 1502 using the aforementioned proprietary CADD Solis NCS communication protocol over Ethernet/WiFi. Firmware and configuration updates can be transferred using a “Best Effort” sub-protocol. In an embodiment, a PLexus Ethernet Firmware Updater (PLEFU) interface applet can be created from Smiths Medical's CADD Solis USN Firmware Updater (SUFU). Irrespective of a particular embodiment, it is to be appreciated and understood that PC 1522 can comprise an “Internet of Things” or “IoT” implementation that can optionally be in or with, for example, one or more separate devices or objects of interest or utility to patient care system 1500.

Axeda PC 1522 is configured to communicate with each pump 1518 using the aforementioned proprietary CADD Solis NCS communication protocol over USB. Each pump 1518 is therefore configured to further implement a standard USB Virtual Serial Port class. In embodiments, the PLexus Usb Firmware Updater (PLUFU) interface applet can be created from Smiths Medical's CADD Solis USB Firmware Updater (SUFU).

In further embodiments, patient care system 1500 further comprises an Axeda enterprise server 1524. Axeda PC 1522 can communicate with Axeda enterprise server 1524 via an HTTPS protocol over the Internet. A firmware or configuration package 1526 can be integrated into external server 1504 or Axeda enterprise server 1524.

In other embodiments, rack 1502 is optional within patient care system 1500. Components of patient care system 1500 such as external server 1504, MSS system 1506, TC unit 1508, customer/third party control system 1510, central display 1512, handheld or mobile device 1514, pump simulator 1516, pumps 1518, PC 1520, and/or Axeda PC 1522 can communicate without rack 1502. In such embodiments, each component of system 1500 that is interfaced to a separate component can be configured for appropriate communication with that component or components.

Embodiments disclosed herein can be used in combination with systems and methods of other inventions. For example, the concepts of WIPO International Application No. PCT/US2015/038309, titled “MEDICAMENT INFUSION SYSTEM AND PUMP ASSEMBLY FOR USE THEREIN” (that is hereby incorporated by reference to the extent that it is not inconsistent with the instant description), and WIPO International Application No. PCT/US16/22322, titled “MEDICAL DEVICE CUSTOMIZATION” (that is hereby incorporated by reference to the extent that it is not inconsistent with the instant description) can each be utilized in part, in total, alone, or in combination with each other, with embodiments of systems and methods for coordinating and controlling infusion pumps as described by example or otherwise contemplated herein. Both of the aforementioned disclosures provide techniques for separating or distinguishing, for example, IV pumps from enteral pumps from neuraxial pumps.

For example, “pump type” knowledge can be used to change the course of action of the embedded server. A “pump type” rule can alert or prevent 1 mL syringes from being used on the same line as 60 mL due to pressure differences. In another example, for large volume pumps, a “pump type” rule can alert or prohibit multiple secondary infusions on separate LVP pumps on the same line.

In another example such “pump type” knowledge can be used to change the course of action of an embedded server. For example, in FIG. 4, assuming 410a-410e are IV pumps, 410f is an enteral pump, and 410g is a neuraxial pump, 410a-410e will most likely be set up, programmed, or operated differently than 410f and 410g. In this example, the embedded server can limit the choices of drugs based on the route of infusion that is particular to the pump. In the event that the user tries to place the wrong disposable (as sensed by, for example, a device or system of the aforementioned “MEDICAMENT INFUSION SYSTEM AND PUMP ASSEMBLY FOR USE THEREIN”) into the incorrect pump (as sensed by a device or system of the aforementioned “MEDICAL DEVICE CUSTOMIZATION”), the embedded server can use this combined knowledge to guide the user to a correct course of treatment. Additionally, in an embodiment, information from a system disclosed in WIPO International Publication No. WO 2013/074717 A1, titled “MEDICAL TUBING DETECTION AND MANAGEMENT” (that is hereby incorporated by reference to the extent that it is not inconsistent with the instant description), can be parsed into the embedded server, thus helping to minimize possibilities of route-of-infusion errors. In this example, the pump is marked for a specific route of infusion, the disposable is “keyed” such that the disposable will only work on the correct pump, and the manifold can determine which pumps are connected together. Further, combining such systems and methods with the systems and methods described in WIPO International Publication No. WO 2014/210465 A1, titled “INFUSION PLANNING SYSTEM” (that is hereby incorporated by reference to the extent that it is not inconsistent with the instant description), and WIPO International Application No. PCT/US15/63710, titled “INFUSION PLANNING SYSTEM WITH CLINICAL DECISION SUPPORT” (that is hereby incorporated by reference to the extent that it is not inconsistent with the instant description), as well as WIPO International Publication No. WO 2014/116832 A1, titled “MEDICATION SAFETY DEVICES AND METHODS” (that is hereby incorporated by reference to the extent that it is not inconsistent with the instant description) can advantageously, for example, generate or implement systems and methods that effectively “learn” to ensure patient safety.

Irrespective of a particular embodiment, it is to be appreciated and understood that the novel and inventive embedded real-time server devices, systems, and methods—as described by example or otherwise contemplated herein—can (i) provide relatively quick and reliable response times compared to known systems, (ii) be capable of handling multiple hits on a network without delays and network overloads, and (iii) be reliable enough to control infusion pumps on their own. Also, the embedded real-time server devices, systems, and methods, as described by example or otherwise contemplated herein, can provide desired functionality of communicatively coupled devices specifically with respect to their connections to each other; and such devices, systems, and methods can provide a desirable increase in overall pump “intelligence” as compared to, for example, individual pumps deployed for a single patient that are not so communicatively coupled. Additionally, in particular, the embedded real-time server devices, systems, and methods—as described by example or otherwise contemplated herein—can assist in effectively and efficiently managing multiple medical data systems pertaining to a single patient (e.g., Electronic Medical Record (EMR), Hospital Information System (HIS), and Information Technology (IT) systems of the various medical devices) that are communicatively coupled and thereby function with respect to each other. Further, the embedded real-time server devices, systems, and methods, as described by example or otherwise contemplated herein, can provide a useful tool for epidemiology, health management, billing and insurance coverage determinations, and clinical purposes generally, due to the relatively expeditious and efficient interconnected communication pathways of such devices, systems, and methods as compared to, for example, individual pumps deployed for a single patient that are not so communicatively coupled.

Various embodiments of systems, devices, and methods have been described herein. These embodiments are given only by way of example and are not intended to limit the scope of the subject matter hereof. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the subject matter hereof.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A real-time embedded server system for controlling, in real-time, at least one infusion pump, the embedded server system comprising:
a plurality of infusion pumps, wherein each of the infusion pumps includes a pumping mechanism and programmable circuitry configured to receive control commands and control operation of the pumping mechanism;
an embedded server including a memory and a processor electrically coupled to the memory and configured to implement:
a control engine configured to issue control commands to at least one infusion pump, the control commands related to the operation of the at least one infusion pump,
a messaging engine configured to issue messages to and receive messages from the at least one infusion pump,
an aggregation engine configured to aggregate data related to the operation of the at least one infusion pump, and
a networking engine; and
a network operably coupling the plurality of infusion pumps and the embedded server, wherein the networking engine is configured to control network access of the embedded server and the at least one infusion pump.

2. The real-time embedded server system of claim 1, wherein the plurality of infusion pumps each include controls for controlling operation of the pumping mechanism, and wherein the controls are overridden by control commands issued by the control engine.

3. The real-time embedded server system of claim 1, wherein the networking engine is further configured to communicate with a hospital information system.

4. The real-time embedded server system of claim 1, wherein the network is at least one of closed-circuit or private.

5. The real-time embedded server system of claim 1, wherein the networking engine is configured to assign network identifiers to each of the plurality of infusion pumps.

6. The real-time embedded server system of claim 1, further comprising an infusion pump rack, the infusion pump rack comprising a body, a plurality of pump mounting portions, and at least one server mounting portion, wherein each of the plurality of infusion pumps is mounted to one of the plurality of pump mounting portions and the embedded server is mounted to the at least one server mounting portion.

7. The real-time embedded server system of claim 1, wherein the memory comprises a database configured to store the control commands, the messages, the data related to the operation of the at least one infusion pump, and network data.

8. The real-time embedded server system of claim 1, wherein the control engine is configured to issue a control command related to at least one of a piggybacking mode and a takeover mode.

9. The real-time embedded server system of claim 1, wherein at least one of the control engine, messaging engine, and networking engine utilize the aggregation engine to aggregate data related to the operation of the at least one infusion pump.

10. The real-time embedded server system of claim 1, wherein the embedded server is located at a patient's bedside.

11. A method for controlling a plurality of infusion pumps with a real-time embedded server, the method comprising:
implementing a real-time embedded server, the embedded server including a control engine, the control engine configured to issue control commands to at least one infusion pump, the control commands related to the operation of the at least one infusion pump;
implementing at least one infusion pump, the at least one infusion pump configured to receive control commands from the embedded server;
implementing a network operably coupling the embedded server and the at least one infusion pump;
issuing a control command from the control engine to the at least one infusion pump; and
executing an operational mode of the at least one infusion pump based on the control command.

12. The method for controlling a plurality of infusion pumps with the real-time embedded server of claim 11, further comprising receiving a message from the at least one infusion pump.

13. The method for controlling a plurality of infusion pumps with the real-time embedded server of claim 12, wherein the message comprises at least one of a confirmation of the control command, a status, or operational data of the at least one infusion pump.

14. The method for controlling a plurality of infusion pumps with the real-time embedded server of claim 13, further comprising:
issuing a secondary control command from the control engine to the at least one infusion pump, the secondary control command based on the received message.

15. The method for controlling a plurality of infusion pumps with the real-time embedded server of claim 14, further comprising executing a secondary operational mode of the at least one infusion pump based on the secondary control command.

16. A method of aggregating data related to a plurality of infusion pumps with a real-time embedded server, the method comprising:
receiving, with a real-time embedded server, a first data from a first infusion pump, the first data comprising data related to the operation of the first infusion pump;
storing the received first data;
receiving, with an embedded server, a second data from a second infusion pump, the second data comprising data related to the operation of the second infusion pump;
storing the received second data; and
performing an aggregation algorithm on the stored first data and the stored second data.

17. The method of aggregating data related to a plurality of infusion pumps with the real-time embedded server of claim 16, further comprising transmitting an output of the aggregation algorithm to an outside system.

18. The method of aggregating data related to a plurality of infusion pumps with the real-time embedded server of claim 17, wherein the outside system comprises a hospital information system.

19. The method of aggregating data related to a plurality of infusion pumps with the real-time embedded server of claim 16, wherein the first data or the second data is at least one of a message, operational data, or network data.

20. The method of aggregating data related to a plurality of infusion pumps with the real-time embedded server of claim 16, wherein storing the received first data or storing the received second data comprises storing the first data or the second data in a database.

21.-30. (canceled)
